(19) United States
(12) Patent Application Publication
(10) Pub. No.: US 2020/0061103 A1
(43) Pub. Date: Feb. 27, 2020

(54) INHIBITION OF NEUROLOGICAL DISEASE

(71) Applicant: Revalesio Corporation, Tacoma, WA (US)

(72) Inventors: Andreas Kalmes, Tacoma, WA (US); Supurna Ghosh, Tacoma, WA (US)

(21) Appl. No.: 16/597,703

(22) Filed: Oct. 9, 2019

Related U.S. Application Data
(63) Continuation of application No. PCT/US2019/033808, filed on May 23, 2019.
(60) Provisional application No. 62/676,708, filed on May 25, 2018.
(60) Provisional application No. 62/758,384, filed on Nov. 9, 2018.
(60) Provisional application No. 62/758,415, filed on Nov. 9, 2018.

(51) Int. Cl. A61K 33/00; A61K 9/00; A61K 9/16; A61K 9/08; A61P 25/28; A61P 11/00

(52) U.S. Cl. CPC A61K 33/00; A61K 9/0019; A61P 11/00; A61K 9/08; A61P 25/28; A61K 9/16

(57) ABSTRACT

Some embodiments herein include methods of ameliorating, inhibiting, reducing, treating, slowing the progression of, or preventing a decline in respiratory function associated with a neurological disease of a patient, for example amyotrophic lateral sclerosis (ALS). Some embodiments herein include methods of ameliorating, inhibiting, reducing the symptoms of, treating, slowing the progression of, or preventing a neurological disease of a patient, in which the neurological disease comprises a decline in respiratory function. Some embodiments include methods of deferring an ALS intervention in an ALS patient.

### CROSS-REFERENCE TO RELATED APPLICATIONS

The present application claims the benefit of U.S. Provisional Application No. 62/676,708, filed May 25, 2018; U.S. Provisional Application No. 62/758,384, filed Nov. 9, 2018; and U.S. Provisional Application No. 62/758,415, filed Nov. 9, 2018, each of which is incorporated by reference in its entirety herein.

### BACKGROUND

Respiratory function can be impaired in neuromuscular disease, for example, neuromuscular diseases such as amyotrophic lateral sclerosis (ALS), Duchenne's muscular dystrophy (DMD), spinal muscular atrophy (SMA), and Huntington's disease (HD), and also in other neurological diseases such as Alzheimer's disease (AD), Parkinson's disease (PD), and multiple sclerosis (MS). Impaired muscular strength associated with neuromuscular disease can lead to respiratory failure. For example, weakness of inspiratory muscle strength can impair alveolar ventilation, while weakness of expiratory muscles can cause inadequate clearance of airway secretions. Respiratory failure is among the most common causes of morbidity and mortality in patients with neuromuscular disease (See Racca et al. (2010), “Respiratory management of acute respiratory failure in neuromuscular diseases” Minerva Anestesiologica 76: 51-62).

ALS is a degenerative neurological disease characterized by loss of motor neurons. Symptoms include loss of control of voluntary muscles, weakening of these muscles, and spinal muscular atrophy. This can result in difficulty speaking, swallowing, and breathing.

Immunological components have been implicated in ALS. Different types of immune cells, including macrophages, neutrophils, T cells, astrocytes, and microglia, can contribute to the pathology of immune-related diseases like ALS. In addition, pro-inflammatory cytokines (specifically TNF-alpha) have been found to be elevated in Alzheimer's disease, Parkinson's disease, and ALS. (Greig et al (2006) Ann NY Acad of Sci 1035:290-315.)

### FIELD

Some embodiments herein relate to methods and uses of oxygenated ionic aqueous solution for ameliorating, inhibiting, reducing the symptoms of, treating, and/or slowing the progression of neurological diseases that comprise a decline in respiratory function, such as neuromuscular diseases (e.g., ALS).

### SUMMARY

Some embodiments include a method of ameliorating, inhibiting, reducing, treating, slowing the progression of, or preventing a decline in respiratory function (or the rate of decline of respiratory function) associated with a neurological disease of a patient. The method can comprise selecting the patient as having the neurological disease and undergoing a decline in respiratory function, having the neurological disease and at risk of a decline in respiratory function, at risk of the neurological disease and undergoing a decline in respiratory function, or at risk of the neurological disease and at risk of undergoing a decline in respiratory function. The method can comprise administering an effective amount of oxygenated ionic aqueous solution to the patient, thereby ameliorating, inhibiting, reducing, treating, slowing the progression of, or preventing the decline in respiratory function (or the rate of decline of respiratory function) associated with the neurological disease. In some embodiments, the oxygenated ionic aqueous solution is a pharmaceutical saline solution comprising stabilized oxygen-containing nanostructures, a majority of the nanostructures having a diameter of less than 100 nanometers, wherein the pharmaceutical saline solution comprised at least 20 ppm oxygen at the time it was manufactured. In some embodiments, the neurological disease is selected from the group consisting of amyotrophic lateral sclerosis (ALS), Duchenne's muscular dystrophy (DMD), spinal muscular atrophy (SMA) (such as SMA type I, SMA type II, or SMA type III), Huntington's disease (HD), Alzheimer's disease (AD), and Parkinson's disease (PD). In some embodiments, the neurological disease is selected from the group consisting of DMD, SMA (such as SMA type I, SMA type II, or SMA type III), HD, AD, and PD. In some embodiments, the neurological disease is selected from the group consisting of ALS, DMD, SMA (such as SMA type I, SMA type II, or SMA type III), HD, and PD. In some embodiments, the neurological disease is selected from the group consisting of DMD, SMA (such as SMA type I, SMA type II, or SMA type III), HD, and PD. In some embodiments, the neurological disease is selected from the group consisting of ALS, DMD, SMA (such as SMA type I, SMA type II, or SMA type III), HD, AD, PD, and MS. In some embodiments, the neurological disease is selected from the group consisting of DMD, SMA (such as SMA type I, SMA type II, or SMA type III), HD, AD, PD, and MS. In some embodiments, the neurological disease is selected from the group consisting of ALS, DMD, SMA (such as SMA type I, SMA type II, or SMA type III), and HD. In some embodiments, the neurological disease is selected from the group consisting of ALS, DMD, SMA (such as SMA type I, SMA type II, or SMA type III), and HD. In some embodiments, the neurological disease is selected from the group consisting of DMD, SMA (such as SMA type I, SMA type II, or SMA type III), and HD. In some embodiments, the neurological disease is selected from the group consisting of ALS, DMD and SMA (such as SMA type I, SMA type II, or SMA type III). In some embodiments, the neurological disease is selected from the group consisting of DMD and SMA (such as SMA type I, SMA type II, or SMA type III). In some embodiments, the neurological disease is ALS. In some embodiments, the patient does not have ALS. In some embodiments, the oxygenated ionic aqueous solution is administered to the subject repeatedly over a period of weeks. The administering can comprise intravenous administration, inhalation, or intravenous administration and inhalation. In some embodiments, the method further comprises deferring use of a ventilator for the patient for at least 1 week following said administering. In some embodiments, in the absence of the administering, the ventilator would have been expected at a time, and wherein the ventilator is deferred beyond the expected time. In some embodiments, the ventilator is deferred for at least 1 week, 2 weeks, 3 weeks, 4 weeks, 5 weeks, 6 weeks, 7 weeks, 8 weeks, 9 weeks, 10 weeks, 11 weeks, 12 weeks, or for 1-2 weeks, 1-3 weeks, 1-4 weeks, 1-5 weeks, 1-6 weeks, 1-7 weeks, 1-8 weeks, 1-9 weeks, 1-10 weeks, 1-11 weeks, 1-12 weeks, 2-3 weeks, 2-4 weeks, 2-5 weeks, 2-6 weeks, 2-7 weeks, 2-8 weeks, 2-9 weeks, 2-10 weeks, 2-11 weeks, 2-12 weeks, 3-4 weeks, 3-5 weeks, 3-6 weeks, 3-7 weeks, 3-8 weeks, 3-9 weeks, 3-10 weeks, 3-11 weeks, 3-12 weeks, 4-5 weeks, 4-6 weeks, 4-7 weeks, 4-8 weeks, 4-9 weeks, 4-10 weeks, 4-11 weeks, 4-12 weeks, 5-6 weeks, 5-7 weeks, 5-8 weeks, 5-9 weeks, 5-10 weeks, 5-11 weeks, 5-12 weeks, 6-7 weeks, 6-8 weeks, 6-9 weeks, 6-10 weeks, 6-11 weeks, or 6-12 weeks. In some embodiments, the method further comprises detecting an inhibition or reduction or decline in vital capacity in the patient following said administration. In some embodiments, the decline in respiratory function comprises, consists essentially of, or consists of a decline in vital capacity. In some embodiments, ameliorating, inhibiting, reducing, treating, slowing the progression of, or preventing the decline of respiratory function (or the rate of decline of respiratory function) comprises reducing a rate at which vital capacity declines, halting a decline in vital capacity, or increasing vital capacity. In some embodiments, ameliorating, inhibiting, reducing, treating, slowing the progression of, or preventing the decline of respiratory function (or the rate of decline of respiratory function) comprises reducing a rate at which vital capacity declines or halting a decline in vital capacity. In some embodiments, the vital capacity comprises SVC and/or FVC. In some embodiments, the patient is selected as having the neurological disease and undergoing the decline in respiratory function or having the neurological disease and being at risk of the decline in respiratory function. In some embodiments, the patient is selected as being at risk of the neurological disease and being at risk of the decline in respiratory function, or being at risk of the neurological disease and undergoing the decline in respiratory function. In some embodiments, the patient is selected as being at risk of the neurological disease and undergoing the decline in respiratory function, or having the neurological disease and undergoing the decline in respiratory function. In some embodiments the patient is selected as being at risk of the neurological disease and being at risk of the decline in respiratory function, or having the neurological disease and being at risk of the decline in respiratory function.

Some embodiments include a method of ameliorating, inhibiting, reducing the symptoms of, treating, slowing the progression of, or preventing a neurological disease of a patient or a symptom thereof. The neurological disease can comprise a decline in respiratory function. The method can comprise administering an amount of oxygenated ionic aqueous solution to the patient in an amount effective to ameliorate, inhibit, reduce, treat, slow the progression of, or prevent the decline in respiratory function in the patient. Thus, the neurological disease can be ameliorated, inhibited, treated, slowed in progression, or prevented. In some embodiments, the oxygenated ionic aqueous solution is a pharmaceutical saline solution comprising stabilized oxygen-containing nanostructures, a majority of the nanostructures having a diameter of less than 100 nanometers, wherein the pharmaceutical saline solution comprised at least 20 ppm oxygen at the time it was manufactured. In some embodiments, the amount of oxygenated ionic aqueous solution is effective to inhibit the decline in respiratory function. In some embodiments, the administering comprises administering the oxygenated ionic aqueous solution to the patient repeatedly over a period of weeks, said administering comprising intravenous administration, inhalation, or intravenous administration and inhalation. In some embodiments, the method further comprises selecting the patient as having, or being at risk of having neurological disease comprising the decline in respiratory function. In some embodiments, the method further comprises deferring use of a ventilator for the patient for at least 1 week following said administering. In some embodiments, in the absence of the administering of oxygenated ionic aqueous solution, the ventilator would have been expected at a time, and the ventilator is deferred beyond the expected time. In some embodiments, the ventilator is deferred for at least 1 week, 2 weeks, 3 weeks, 4 weeks, 5 weeks, 6 weeks, 7 weeks, 8 weeks, 9 weeks, 10 weeks, 11 weeks, 12 weeks, including ranges between any two of the listed values, for example 1-2 weeks, 1-3 weeks, 1-4 weeks, 1-5 weeks, 1-6 weeks, 1-7 weeks, 1-8 weeks, 1-9 weeks, 1-10 weeks, 1-11 weeks, 1-12 weeks, 2-3 weeks, 2-4 weeks, 2-5 weeks, 2-6 weeks, 2-7 weeks, 2-8 weeks, 2-9 weeks, 2-10 weeks, 2-11 weeks, 2-12 weeks, 3-4 weeks, 3-5 weeks, 3-6 weeks, 3-7 weeks, 3-8 weeks, 3-9 weeks, 3-10 weeks, 3-11 weeks, 3-12 weeks, 4-5 weeks, 4-6 weeks, 4-7 weeks, 4-8 weeks, 4-9 weeks, 4-10 weeks, 4-11 weeks, 4-12 weeks, 5-6 weeks, 5-7 weeks, 5-8 weeks, 5-9 weeks, 5-10 weeks, 5-11 weeks, 5-12 weeks, 6-7 weeks, 6-8 weeks, 6-9 weeks, 6-10 weeks, 6-11 weeks, or 6-12 weeks. In some embodiments, the neurological disease is selected from the group consisting of ALS, DMD, SMA (such as SMA type I, SMA type II, or SMA type III), Huntington's disease, AD, and PD. In some embodiments, the neurological disease is selected from the group consisting of DMD, SMA (such as SMA type I, SMA type II, or SMA type III), Huntington's disease, AD, and PD. In some embodiments, the neurological disease is selected from the group consisting of ALS, DMD and SMA (such as SMA type I, SMA type II, or SMA type III). In some embodiments, the neurological disease is selected from the group consisting of DMD and SMA (such as SMA type I, SMA type II, or SMA type III). In some embodiments, the patient does not have ALS. In some embodiments, ameliorating, inhibiting, reducing, treating, slowing the progression of, or preventing the decline of respiratory function (or the rate of decline of respiratory function) comprises reducing a rate at which vital capacity declines or halting a decline in vital capacity. In some embodiments, the vital capacity comprises SVC and/or FVC.

Some embodiments include a method of inhibiting, treating, preventing, ameliorating, or reducing the symptoms of ALS. The method can comprise administering an oxygenated ionic aqueous solution to an ALS patient repeatedly over a period of weeks. The administering can comprise intravenous administration, inhalation, or intravenous administration and inhalation. The method can comprise deferring an ALS intervention in the patient for at least 1 week following the administering. In some embodiments, the method further comprises ameliorating the decline of vital capacity (e.g., SVC and/or FVC) in the ALS patient following said administration. In some embodiments, the method further comprises ameliorating, inhibiting, reducing, treating, slowing the progression of, or preventing a decline of respiratory function (or the rate of decline of respiratory function), e.g., vital capacity as measured by SVC and/or FVC, in the ALS patient following said administration. By way of example, the patient can be selected as undergoing or being at risk of a decline in respiratory function, and in need of a treatment, amelioration, inhibition, reduction, slowing of the rate of progression of, or prevention of the decline in respiratory function (or the rate of decline in respiratory function). It is noted that when terms such as “inhibiting,” “reducing,” and “slowing” (including variations of these root terms) are mentioned herein in connection with the decline in respiratory function, also expressly contemplated is inhibiting, reducing, slowing the rate of decline in respiratory function. Examples of ameliorating, inhibiting, reducing, treating, slowing the progression of, or preventing the decline in respiratory function include reducing the rate at which vital capacity declines (such as reducing the rate of decline in SVC and/or FVC), halting a decline in vital capacity, and/or improving vital capacity. In some embodiments, ameliorating, inhibiting, reducing, treating, slowing progression of, or preventing the decline of respiratory function (or the rate of decline of respiratory function) comprises reducing the rate at which vital capacity declines, halting a decline in vital capacity, or increasing vital capacity. In some embodiments, ameliorating, inhibiting, reducing, treating, slowing the progression of, or preventing the decline of respiratory function (or the rate of decline of respiratory function) comprises reducing the rate at which vital capacity declines or halting a decline in vital capacity. In some embodiments, ameliorating, inhibiting, reducing, treating, slowing the progression of; or preventing the decline of respiratory function (or the rate of decline of respiratory function) comprises reducing the rate at which vital capacity declines. In some embodiments, ameliorating, inhibiting, reducing, treating, slowing the progression of, or preventing the decline of respiratory function (or the rate of decline of respiratory function) comprises halting a decline in vital capacity or increasing vital capacity. In some embodiments, the method further comprises detecting an amelioration of the decline of vital capacity (e.g., SVC and/or FVC) in the ALS patient following said administration. In some embodiments, for any method described herein, the ALS intervention comprises use of a ventilator and/or a tracheotomy. In the method of some embodiments, the oxygenated ionic aqueous solution is a pharmaceutical saline solution comprising stabilized oxygen-containing nanostructures (e.g., nanobubbles), a majority of the nanostructures (e.g., nanobubbles) having a diameter of less than 100 nanometers, in which the pharmaceutical saline solution comprised at least 20 ppm oxygen at the time it was manufactured, for example at least 20 ppm, 30 ppm, 40 ppm, 50 ppm, or 60 ppm oxygen, including ranges between any two of the listed values. In some of the method embodiments, in the absence of administration of the oxygenated ionic aqueous solution, the ALS intervention would have been expected at a time, and in accordance with the method comprising administration of the oxygenated ionic aqueous solution, the ALS intervention is deferred beyond the expected time. In the method of some embodiments, the ALS intervention is deferred for at least 2 weeks, 3 weeks, 4 weeks, 5 weeks, 6 weeks, 7 weeks, 8 weeks, 9 weeks, 10 weeks, 11 weeks, or 12 weeks. In the method of some embodiments, the ALS intervention is deferred for 1-12 weeks, such as 1-2 weeks, 1-3 weeks, 1-4 weeks, 1-5 weeks, 1-6 weeks, 1-7 weeks, 1-8 weeks, 1-9 weeks, 1-10 weeks, 1-11 weeks, 1-12 weeks, 2-3 weeks, 2-4 weeks, 2-5 weeks, 2-6 weeks, 2-7 weeks, 2-8 weeks, 2-9 weeks, 2-10 weeks, 2-11 weeks, 2-12 weeks, 3-4 weeks, 3-5 weeks, 3-6 weeks, 3-7 weeks, 3-8 weeks, 3-9 weeks, 3-10 weeks, 3-11 weeks, 3-12 weeks, 4-5 weeks, 4-6 weeks, 4-7 weeks, 4-8 weeks, 4-9 weeks, 4-10 weeks, 4-11 weeks, 4-12 weeks, 5-6 weeks, 5-7 weeks, 5-8 weeks, 5-9 weeks, 5-10 weeks, 5-11 weeks, 5-12 weeks, 6-7 weeks, 6-8 weeks, 6-9 weeks, 6-10 weeks, 6-11 weeks, or 6-12 weeks. In some embodiments of the method, treatment with the oxygenated aqueous ionic solution can increase survival of the ALS patient. The survival can be increased beyond a time that would have been expected if the ALS patient had not received the oxygenated aqueous ionic solution.

Some embodiments include a method of deferring an ALS intervention in an ALS patient. The method can comprise identifying the ALS patient as in need of the ALS intervention at a future time if the patient is untreated with an oxygenated ionic aqueous solution. The method can comprise administering the oxygenated ionic aqueous solution to the ALS patient repeatedly over a period of weeks. The administration can comprise intravenous administration, inhalation, or intravenous administration and inhalation. Accordingly, the ALS intervention can be deferred beyond the future time by at least 1 week. In some embodiments, the method further comprises detecting an inhibition of decline in vital capacity (e.g., SVC and/or FVC) in the ALS patient following said administration. In the method of some embodiments, the ALS intervention comprises use of a ventilator and/or a tracheotomy. In the method of some embodiments, the oxygenated ionic aqueous solution is a pharmaceutical saline solution comprising stabilized oxygen-containing nanostructures (e.g., nanobubbles), a majority of the nanostructures (e.g., nanobubbles) having a diameter of less than 100 nanometers, in which the pharmaceutical saline solution comprised at least 20 ppm oxygen at the time it was manufactured. In the method of some embodiments, the ALS intervention is deferred beyond the future time for at least 2 weeks, such as at least 3 weeks, 4 weeks, 5 weeks, 6 weeks, 7 weeks, 8 weeks, 9 weeks, 10 weeks, 11 weeks, or 12 weeks. In the method of some embodiments, the ALS intervention is deferred beyond the future time for 1-12 weeks, such as 1-2 weeks, 1-3 weeks, 1-4 weeks, 1-5 weeks, 1-6 weeks, 1-7 weeks, 1-8 weeks, 1-9 weeks, 1-10 weeks, 1-11 weeks, 1-12 weeks, 2-3 weeks, 2-4 weeks, 2-5 weeks, 2-6 weeks, 2-7 weeks, 2-8 weeks, 2-9 weeks, 2-10 weeks, 2-11 weeks, 2-12 weeks, 3-4 weeks, 3-5 weeks, 3-6 weeks, 3-7 weeks, 3-8 weeks, 3-9 weeks, 3-10 weeks, 3-11 weeks, 3-12 weeks, 4-5 weeks, 4-6 weeks, 4-7 weeks, 4-8 weeks, 4-9 weeks, 4-10 weeks, 441 weeks, 4-12 weeks, 5-6 weeks, 5-7 weeks, 5-8 weeks, 5-9 weeks, 5-10 weeks, 5-11 weeks, 5-12 weeks, 6-7 weeks, 6-8 weeks, 6-9 weeks, 6-10 weeks, 6-11 weeks, or 6-12 weeks. In the method of some embodiments, the period of weeks is at least about 10 weeks, such as at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 weeks.

The following are contemplated in accordance with the methods of any of the embodiments described herein, including, without limitation, any of the methods of the preceding paragraphs. In some embodiments, for any of the methods described herein, the period of weeks is at least about 4 weeks, such as at least about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 weeks. In some embodiments, for any of the methods described herein, the period of weeks is at least about 11 weeks. In some embodiments, for any of the methods described herein, the period of weeks is at least about 23 weeks. In some embodiments, for any of the methods described herein, the period of weeks is about 20-50 weeks. In some embodiments, for any of the methods described herein, the period of weeks is about 10-20 weeks, 10-30 weeks, 10-40 weeks, 10-50 weeks, 10-60 weeks, 20-30 weeks, 20-40 weeks, 20-50 weeks, 20-60 weeks, 30-40 weeks, 30-50 weeks, 30-60 weeks, 40-50 weeks, 40-60 weeks, or 50-60 weeks. In some embodiments, for any of the methods described herein, the oxygenated ionic aqueous solution is administered intravenously weekly over the period of weeks. In some embodiments, for any of the methods described herein, the oxygenated ionic aqueous solution is administered intravenously, in an amount of at least 100 ml, such as at least 100 ml, 150 ml, 200 ml 250 ml, 300 ml, 350 ml, 400 nil, 450 ml, or 500 ml per intravenous administration. In some embodiments, for any of the methods described herein, the oxygenated ionic aqueous solution is administered intravenously in an amount of at least 350 ml per intravenous administration. In some embodiments, for any of the methods described herein, the oxygenated ionic aqueous solution is administered intravenously in an amount of about 100 ml-1000 ml per intravenous administration. In some embodiments, for any of the methods described herein, the oxygenated ionic aqueous solution is administered intravenously in an amount of about 200 ml600 ml per intravenous administration. In some embodiments, for any of the methods described herein, the oxygenated ionic aqueous solution is administered intravenously in an amount of about 300 ml-500 ml per intravenous administration. In some embodiments, for any of the methods described herein, the oxygenated ionic aqueous solution is administered intravenously in an amount of at least 375 ml per intravenous administration. In the method of some embodiments, the oxygenated ionic aqueous solution is administered intravenously in an amount of about 300 ml-500 ml per intravenous administration. In some embodiments, for any of the methods described herein, the oxygenated ionic aqueous solution is administered by inhalation at least 2 times per week over the period of weeks. In some embodiments, for any of the methods described herein, the oxygenated ionic aqueous solution is administered by inhalation at least 4 times per week over the period of weeks. In some embodiments, for any of the methods described herein, the oxygenated ionic aqueous solution is administered by inhalation at least 6 times per week over the period of weeks. In some embodiments, for any of the methods described herein, the oxygenated ionic aqueous solution is administered by inhalation 2 to 12 times per week over the period of weeks. In some embodiments, for any of the methods described herein, the oxygenated ionic aqueous solution is administered by inhalation 4 to 8 times per week over the period of weeks. In some embodiments, for any of the methods described herein, the oxygenated ionic aqueous solution is administered by inhalation in an amount of at least 2 ml. In some embodiments, for any of the methods described herein, the oxygenated ionic aqueous solution is administered by inhalation in an amount of at least 4 ml. In some embodiments, for any of the methods described herein, the oxygenated ionic aqueous solution is administered by inhalation in an amount of about 1 ml to 10 ml. In some embodiments, for any of the methods described herein, the oxygenated ionic aqueous solution is administered by inhalation in an amount of about 2 ml to 8 ml. In some embodiments, for any of the methods described herein, the inhalation comprises nebulization of the oxygenated ionic aqueous solution. In some embodiments, for any of the methods described herein, the oxygenated ionic aqueous solution comprises stabilized oxygen-containing nanostructures, a majority of the nanostructures having a diameter of less than 100 nanometers. In some embodiments, for any of the methods described herein, the oxygenated ionic aqueous solution comprises at least 15 ppm oxygen at standard temperature and pressure, such as at least 20 ppm, 25 ppm, 30 ppm, 35 ppm, 40 ppm, 45 ppm, 50 ppm, 55 ppm, 60 ppm, 65 ppm, 70 ppm, 75 ppm, 80 ppm, or a range such as 15 ppm-70 ppm; 20 ppm-70 ppm; 40 ppm-70 ppm, 15 ppm-60 ppm, 20 ppm-60 ppm, or 40 ppm-60 ppm. In some embodiments, for any of the methods described herein, the oxygenated ionic aqueous solution is a saline solution comprising at least 40 ppm oxygen. In some embodiments, for any of the methods described herein, the oxygenated ionic aqueous solution is a pharmaceutical solution wherein the pharmaceutical saline solution comprised at least 20 ppm oxygen at the time it was manufactured. In some embodiments, for any of the methods described herein, the oxygenated ionic aqueous solution comprises saline. In some embodiments, for any of the methods described herein, the oxygen in the oxygenated ionic aqueous solution comprises modified or charged oxygen species. In some embodiments, for any of the methods described herein, the oxygenated ionic aqueous solution comprises no more than trace amounts of ozone. In some embodiments, for any of the methods described herein, the oxygen in the oxygenated ionic aqueous solution has been present in an amount of at least 15 ppm at standard temperature and pressure (typically 0° C. and 100 kPa) for at least 3 hours. In some embodiments, for any of the methods described herein, the oxygen in the oxygenated ionic aqueous solution has been present in an amount of at least 40 ppm at standard temperature and pressure for at least 3 hours. In some embodiments, for any of the methods described herein, the oxygenated ionic aqueous solution comprises solvated electrons stabilized by molecular oxygen. In some embodiments, for any of the methods described herein, the oxygenated ionic aqueous solution is a pharmaceutical saline solution that comprised at least 20 ppm oxygen at the time it was manufactured (such as at least 30 ppm, 40 ppm, 60 ppm, 70 ppm, or 80 ppm), and is administered intravenously weekly in an amount of at least 200 ml (such as at least 200 ml, 250 ml, 300 ml or 350 ml), and is administered by inhalation comprising nebulization at least 6 times per week in an amount of at least 2 ml (such as at least 1 ml, 2 ml, 3 nil, or 4 nil), and the period of weeks is at least 20 weeks. In some embodiments, for any of the methods described herein, the oxygenated ionic aqueous solution is a pharmaceutical saline solution comprised at least 20 ppm oxygen at the time it was manufactured (such as at least 30 ppm, 40 ppm, 50 ppm, 60 ppm, 70 ppm, or 80 ppm), and is administered intravenously weekly in an amount of at least about 375 ml, and is administered by inhalation comprising nebulization at least 6 times per week in an amount of at least about 4 ml, and wherein the period of weeks is at least 23 weeks. In some embodiments, for any of the methods described herein, the ameliorating, inhibiting, reducing, treating, slowing the progression of, or preventing a decline in respiratory function comprises: enhancing myelination by oligodendrocytes, enhancing myelination by Schwann cells, supporting or enhancing nerve myelination, decreasing motor neuron loss in the spinal cord, preserving neuromuscular junctions, increasing protective function of microglia (such as phagocytosis), activating protective microglia in the central nervous system (e.g., brain and/or spinal cord), or two or more of any of the listed items. In some embodiments, for any of the methods described herein, the method further comprises detecting prevention, amelioration, inhibition, and/or reduction of the symptoms of neurological disease of the subject after treatment with the oxygenated ionic aqueous solution for the period of weeks. In some embodiments, for any of the methods described herein, inhibiting, treating, preventing, ameliorating, or reducing the symptoms of the neurological disease comprises: reduction of peripheral inflammation (effector T cells down and/or regulatory T cells up), reduction of CNS inflammation (decrease in inflammatory glia and/or reduction of NFkB activity), increase in non-inflammatory microglia, increase in survival, maturation and function of oligodendrocytes, enhancement of myelination by Schwann cells, augmentation of neuronal branching, plasticity and neurotransmission, enhancement of neuronal survival (anti-apoptotic), stimulation of mitochondrial biogenesis and function, or two or more of the listed items. In the method of some embodiments, inhibiting, treating, preventing, ameliorating, or reducing the symptoms of ALS comprises: enhancing myelination by oligodendrocytes, enhancing myelination by Schwann cells, supporting or enhancing nerve myelination, decreasing motor neuron loss in the spinal cord, preserving neuromuscular junctions, increasing protective function of microglia (such as phagocytosis), activating protective microglia in the central nervous system (e.g., the brain and/or spinal cord), or two or more of any of the listed items (without being limited by theory, it is contemplated that any combination of the listed items is correlated with, and can result in inhibition, treatment, prevention, amelioration, and/or reduction of the symptoms of ALS). In the method of some embodiments, inhibiting, treating, preventing, ameliorating, or reducing the symptoms of ALS comprises: reduction of peripheral inflammation (effector T cells down and/or regulatory T cells up), reduction of CNS inflammation (e.g., reduction of NFkB activity), and/or stimulation of mitochondrial biogenesis and function, or two or more of the listed items. In the method of some embodiments, the patient is selected as having a deficiency in mitochondrial function, for example impaired mitochondrial respiration. In some embodiments, for any of the methods described herein, the method further comprises detecting at least one of the following after treatment with the oxygenated ionic aqueous solution for the period of weeks: reduction of peripheral inflammation (effector. T cells down and/or regulatory T cells up), and/or reduction of NFkB activity in a sample of the subject, such as a blood sample. In the method of some embodiments, the method further comprises detecting inhibition, treatment, prevention, amelioration, and/or reduction of the symptoms of ALS after treatment with the oxygenated ionic aqueous solution for the period of weeks. In the method of some embodiments, treatment with the oxygenated aqueous ionic solution increases survival of the ALS patient. The survival can be increased beyond a time that would have been expected if the ALS patient had not received the oxygenated aqueous ionic solution. In some embodiments, for any of the methods described herein, treatment with the oxygenated aqueous ionic solution increases survival of the patient. In some embodiments, for any of the methods described herein, the neurological disease comprises at least one of DMD, SMA (such as SMA type I, SMA type II, or SMA type III), HD, AD, PD, MS, poliomyelitis (post-polio syndrome), spino-bulbar muscular atrophy (Kennedy syndrome), GBS, CIDP, CIP, and/or HMSN. In some embodiments, for any of the methods described herein, the neurological disease comprises at least one of DMD, SMA (such as SMA type I, SMA type II, or SMA type III), HD, AD, PD, poliomyelitis (post-polio syndrome), spino-bulbar muscular atrophy (Kennedy syndrome), GBS, CIDP, CIP, and/or HMSN. In some embodiments, for any of the methods described herein, the neurological disease comprises at least one of DMD, SMA (such as SMA type I, SMA type II, or SMA type III), HD, AD, PD, or MS. In some embodiments, for any of the methods described herein, the neurological disease comprises at least one of DMD, SMA (such as SMA type I, SMA type II, or SMA type III), HD, AD, or PD. In some embodiments, for any of the methods described herein, the neurological disease comprises at least one of DMD, SMA (such as SMA type SMA type II, or SMA type III), or HD. In some embodiments, for any of the methods described herein, the neurological disease comprises at least one of DMD, or SMA (such as SMA type I, SMA type II, or SMA type III). In some embodiments, for any of the methods described herein, the neurological disease comprises a neuromuscular disease. In some embodiments, for any of the methods described herein, the neurological disease comprises at least one of ALS, DMD, SMA (such as SMA type I, SMA type II, or SMA type III), HD, AD, PD, or MS. In some embodiments, for any of the methods described herein, the neurological disease comprises at least one of ALS, DMD, SMA (such as SMA type I, SMA type II, or SMA type III), HD, AD, or PD. In some embodiments, for any of the methods described herein, the neurological disease comprises at least one of ALS, DMD, SMA (such as SMA type I, SMA type II, or SMA type III), or HD. In some embodiments, for any of the methods described herein, the neurological disease comprises at least one of ALS, DMD, or SMA (such as SMA type I, SMA type II, or SMA type III).

Some embodiments include a method of ameliorating, inhibiting, reducing, treating, slowing the progression of, or preventing a decline in respiratory function (or the rate of decline of respiratory function) in a patient. The method can comprise selecting the patient as undergoing a decline in respiratory function, or being at risk of undergoing a decline in respiratory function. The method can comprise administering an oxygenated ionic aqueous solution to the patient repeatedly over a period of weeks. The administering can comprise intravenous administration, inhalation, or intravenous administration and inhalation. In some embodiments, the method further comprises deferring use of a ventilator for the patient for at least 1 week following said administering. In some embodiments, the method further comprises detecting an inhibition or reduction of decline in vital capacity in the patient following said administration. In some embodiments, patient is an ALS patient. In some embodiments, the patient is selected as undergoing the decline in respiratory function. In some embodiments, the patient is selected as being at risk of the decline in respiratory function. In some embodiments, the decline in respiratory function comprises, consists essentially of, or consists of a decline in vital capacity. In some embodiments, ameliorating, inhibiting, reducing, treating, slowing the progression of, or preventing the decline of respiratory function (or the rate of decline of respiratory function) comprises reducing a rate at which vital capacity declines, halting a decline in vital capacity, or increasing vital capacity. In some embodiments, ameliorating, inhibiting, reducing, treating, slowing the progression of, or preventing the decline of respiratory function (or the rate of decline of respiratory function) comprises reducing a rate at which vital capacity declines or halting a decline in vital capacity. In some embodiments, the vital capacity comprises, consists essentially of, or consists of SVC and/or FVC. In some embodiments, the vital capacity comprises, consists essentially of, or consists of SVC.

Some embodiments include the oxygenated ionic aqueous solution of any of the methods herein for use in treating the decline in respiratory function associated with a neurological disease of a patient, the use comprising any of the methods herein. Some embodiments include an oxygenated ionic aqueous solution of the methods described herein for use in treating ALS. The use can comprise any of the methods herein.

### BRIEF DESCRIPTION OF THE DRAWINGS

FIG. 1 is a graph illustrating an example curve of dissolved oxygen stability for an oxygenated ionic aqueous solution (RNS60) over time.

FIG. 2 is a schematic representation of an open-label phase IIa study of RNS60 in ALS.

FIG. 3A is a graph of individual patient data for slow vital capacity (SVC). FIG. 3B is a graph of individual patient data for SVC change from baseline.

FIG. 4 is a graph of change from baseline of PET imaging of ¹¹C-PBR28.

FIG. 5A is a graph showing effects of RNS60 in vitro on the number of ensheathing oligodendrocytes. FIG. 5B is a graph showing that RNS60-treated oligodendrocytes had longer ensheathing processes. Data are shown for control 1 and RNS60-treated 2 oligodendrocytes.

FIG. 6A and FIG. 6B are graphs showing that RNS60 supports nerve myelination in ALS mice. CNPase and MBP are components of myelin. FIG. 6A shows CNPase, and FIG. 6B shows MBP.

FIG. 7A is a graph showing RNS60 decreases motor neuron loss in the spinal cord of ALS mice. FIGS. 7B-D are a series of images showing that RNS60 decreases motor neuron loss in the spinal cord of ALS mice. Scale bar is 100 μM FIG. 7E is a graph showing that RNS60 preserves neuromuscular junctions (NMJ) in ALS mice.

FIGS. 8A-F are a series of schematic graphs showing that RNS60 protects multiple components of the neuromuscular system. For each bar graph, the relevant region in the neuromuscular system is shown in an accompanying schematic diagram. Shown are data demonstrating protection of neurons (FIGS. 8A-B), protection of components of myelin (FIG. 8C-D), and protection of neuromuscular junctions or NMJs (FIG. 8E-F).

FIGS. 9A-B are a series of graphs showing that RNS60 increases protective function of microglia (in vitro).

FIGS. 10A-B are a series of graphs showing that RNS60 activates protective microglia in the spinal cord of ALS mice. Shown are levels of IL-4 (a protective cytokine) (FIG. 10A) and Ym1 (a marker of protective (M2) glia) (FIG. 10B).

FIGS. 11A-C are a series of microscopic images showing CD68-positive (protective) microglia in healthy mice (FIG. 11A), ALS control mice (FIG. 11B), and ALS mice treated with RNS60 (FIG. 11C).

FIG. 12 is a graph showing that RNS60 protects ALS patient-derived cells under mitochondrial stress.

### DETAILED DESCRIPTION

Some embodiments relate to oxygenated ionic aqueous solution for use in treating degenerative neurological diseases such as ALS. It is reported herein that oxygenated ionic aqueous solutions can inhibit the decline in vital capacity (e.g., SVC and/or forced vital capacity (FVC)) in subjects having ALS. The rate of decline in vital capacity is associated with clinical events in ALS, including respiratory failure, tracheotomy, and death. It is contemplated herein that in ALS subjects treated with oxygenated ionic aqueous solution according to some embodiments herein, ALS interventions can be deferred, such as tracheotomy or ventilator. It is contemplated that methods according to some embodiments can also defer time of death in subjects having ALS.

Unexpectedly, in a phase IIa study, the rate of decline in vital capacity slowed approximately 5-fold in ALS patients treated with the oxygenated ionic aqueous solution (compared to controls) (See Example 2). To the best of Applicants' knowledge, there is no published data that conventional treatments of ALS, such as riluzole and/or edaravone can reduce the rate of vital capacity decline (See, e.g, Andrews et al., JAMA Neurol. 2018; and Abe et al., Lancet Neurol 2017). Accordingly, it is contemplated herein that the inhibition of vital capacity decline not only demonstrates the efficacy of the oxygenated ionic aqueous solution in inhibiting, treating, and/or reducing the severity of ALS, but furthermore, following treatment with the oxygenated ionic aqueous solution, ALS interventions can be deferred. This can include deferring invasive interventions such as ventilator and tracheotomy, which can be associated with risk of infection, loss of ability to eat, and loss of ability to speak. Accordingly, deferring time to ventilator and/or tracheotomy in accordance with some embodiments herein can be advantageous.

Some embodiments relate to oxygenated ionic aqueous solution for use in ameliorating, inhibiting, reducing, treating, slowing the progression of, or preventing the decline of respiratory function (or the rate of decline of respiratory function) in patients. The respiratory function can be associated with a neurological disease, for example a neuromuscular disease such as ALS, DMD, SMA (type I, type II, and/or type III), and/or HD, as well as in other neurological diseases such as AD, PD, and MS. It is reported herein that oxygenated ionic aqueous solutions can inhibit the decline in vital capacity (e.g., SVC and/or FVC), a well-accepted indicator of respiratory function, in subjects having a neurological disease (See Example 2). Without being limited by theory, it is contemplated that the oxygenated ionic aqueous solution has neuroprotective effects, for example on motor neurons and neuromuscular junctions as well as supporting glial cells. As such, the oxygenated aqueous solution of some embodiments is contemplated to be useful for treating, ameliorating, inhibiting reducing, treating, slowing the progression of, or preventing neurological disease (or symptoms thereof) having an associated decline in respiratory function such as SVC and/or FVC. It is noted that when terms such as “inhibiting,” “reducing,” and “slowing” (including variations of these root terms) are mentioned herein in connection with the decline in respiratory function, also expressly contemplated is inhibiting, reducing, slowing the rate of decline in respiratory function. In some embodiments, patients suffering from, or at risk of neurological disease and/or decline in respiratory function associated with the neurological disease, can be treated with oxygenated ionic aqueous solution, interventions such as ventilators can be deferred. It is contemplated that methods according to some embodiments can also defer time of death in subjects having neurological disease as described herein.

### Neurological Disease

In a phase Ba study, the rate of decline in vital capacity slowed approximately 5-fold in ALS patients treated with the oxygenated ionic aqueous solution (compared to controls) (See Example 2). To the best of Applicants' knowledge, there is no published data that conventional treatments of ALS, such as riluzole and/or edaravone can reduce the rate of vital capacity decline (See, e.g, Andrews et al., JAMA Neurol. 2018; and Abe et al., Lancet Neurol 2017). Furthermore, a number of neuromuscular diseases are associated with a decline in respiratory function. For example, slowly progressive motor neuron diseases such as SMA (types I, II, and II) can cause acute exacerbation of chronic respiratory failure (Racca et al., Minerva Anestesiologica 76: 51-62). PD patients have been reported to have lower respiratory pressure than controls, even in early stages of the disease, but these respiratory changes appear to be unrelated to dopaminergic dysfunction (Guedes et al. (2012), Arq. Neuropsiquiatr 70: 847-851), suggesting that the respiratory changes may be associated with motor neurons, but not require intervention at the level of dopaminergic neurons. Similarly, in Huntington's disease, dysregulation of the respiratory center results in irregular breathing patterns, decreases muscle strength, and lung volumes (Jones et al. (2016), “Respiratory function in Huntington's disease should be monitored from middle stage to preclude respiratory failure” Eur. Respir. J. doi: 10.1183/13993003.02215-2015 pp. 1-3). DMD causes progressive muscular weakness, and decline in FVC is associated with the progression of DMD. Indeed, FVC is considered a significant clinical parameter to monitor in the course of the evolution of DMD (Khirani et al. (2014), “Respiratory Muscle Decline in Duchenne Muscular Dystrophy” Pediatric Pulmonology 49:473-481). ALS is also associated with reduced respiratory function, and vital capacity is an indicator of survival and disease progression in ALS patients (See, e.g, Czaplinski et al., J Neurol Neurosurg Psychiatry 2006). Thus, it will be appreciated that neuroprotective effects, for example, protection of motor neurons, neuromotor junctions and/or glial cells supporting motor neurons can inhibit the decline in respiratory function associated with neurological diseases, and can be useful in ameliorating, inhibiting, reducing the symptoms of, treating, slowing the progression of, or preventing a neurological disease, such as a neuromuscular disease. As shown herein in Example 2, oxygenated ionic aqueous solutions in accordance with some embodiments inhibited the rate of decline of slow vital capacity. Furthermore, as shown in Examples 3-8, the oxygenated ionic aqueous solutions in accordance with some embodiments had protective effects on components of the neuromuscular system both on oligodendrocyte cultures in vitro (Example 3) and in a murine ALS model (Examples 4-8). Without being limited by theory, it is contemplated that oxygenated ionic aqueous solutions in accordance with some embodiments can inhibit, reduce, treat, prevent, or slow the progression of, decline of respiratory function (or the rate of decline of respiratory function) in neurological disease, for example through neuroprotective effects on motor neurons and neuromuscular junctions (See Example 6) and their supporting glia. Accordingly, in some embodiments, oxygenated ionic aqueous solution can ameliorate, inhibit, reduce the symptoms of, treat, slow the progression of, or prevent a neurological disease in a patient. Examples of neurological diseases include, but are not limited to ALS, DMD, SMA (such as SMA type I, SMA type II, or SMA type III), HD, AD, PD, and MS. In some embodiments, the neurological disease is selected from the group consisting of ALS, DMD, SMA (such as SMA type I, SMA type II, or SMA type III), HD, AD, and PD, including two or more of the listed items. In some embodiments, the neurological disease is selected from the group consisting of ALS, DMD, SMA (such as SMA type I, SMA type II, or SMA type III), AD, and PD, including two or more of the listed items. In some embodiments, the neurological disease is selected from the group consisting of ALS, DMD, SMA (such as SMA type I, SMA type II, or SMA type III), HD, and PD, including two or more of the listed items. In some embodiments, the neurological disease is selected from the group consisting of ALS, DMD, SMA (such as SMA type I, SMA type II, or SMA type III), AD and PD, including two or more of the listed items. In some embodiments, the neurological disease is selected from the group consisting of ALS, DMD, SMA (such as SMA type I, SMA type II, or SMA type III), HD and MS, including two or more of the listed items. In some embodiments, the neurological disease is selected from the group consisting of ALS, DMD, SMA (such as SMA type I, SMA type II, or SMA type III), MS, AD, and PD, including two or more of the listed items. In some embodiments, the neurological disease is selected from the group consisting of ALS, MS, HD, AD, and PD, including two or more of the listed items. In some embodiments, the neurological disease is selected from the group consisting of ALS, MS, AD, and PD, including two or more of the listed items. In some embodiments, the neurological disease is selected from the group consisting of DMD, SMA (such as SMA type I, SMA type II, or SMA type III), HD, AD, PD, and MS. In some embodiments, the neurological disease is selected from the group consisting of DMD, SMA (such as SMA type I, SMA type II, or SMA type III), HD, AD, and PD, including two or more of the listed items. In some embodiments, the neurological disease is selected from the group consisting of DMD, SMA (such as SMA type I, SMA type II, or SMA type III), AD, and PD, including two or more of the listed items. In some embodiments, the neurological disease is selected from the group consisting of DMD, SMA (such as SMA type I, SMA type II, or SMA type III), HD, and PD, including two or more of the listed items. In some embodiments, the neurological disease is selected from the group consisting of DMD, SMA (such as SMA type I, SMA type II, or SMA type III), AD and PD, including two or more of the listed items. In some embodiments, the neurological disease is selected from the group consisting of DMD, SMA (such as SMA type I, SMA type II, or SMA type III), HD and MS, including two or more of the listed items. In some embodiments, the neurological disease is selected from the group consisting of DMD, SMA (such as SMA type I, SMA type II, or SMA type III), MS, AD, and PD, including two or more of the listed items. In some embodiments, the neurological disease is selected from the group consisting of MS, HD, AD, and PD, including two or more of the listed items. In some embodiments, the neurological disease is selected from the group consisting of MS, AD, and PD, including two or more of the listed items. In some embodiments, the neurological disease is a neuromuscular disease such as ALS, DMD, SMA (such as SMA type I, SMA type II, or SMA type III), and/or HD. Vital capacity (such as SVC and/or FVC) can represent an indicator of respiratory function.

It is contemplated that the inhibition of vital capacity decline not only demonstrates the efficacy of the oxygenated ionic aqueous solution in neurological diseases such as DMD, SMA such as SMA type I (severe) SMA type II (intermediate), or SMA type III (mild), HD, AD, PD, and MS, but furthermore, following treatment with the oxygenated ionic aqueous solution (in which the decline of respiratory function is slowed), ventilator treatment can be deferred. A ventilator can be associated with risk of infection, loss of ability to eat, and loss of ability to speak. Accordingly, deferring time to ventilator in accordance with some embodiments herein can be advantageous.

It is contemplated that oxygenated ionic aqueous solutions can be useful for ameliorating, inhibiting, reducing, treating, slowing the progression of, or preventing a decline in respiratory function associated with a neurological disease in methods, uses, and medicaments of some embodiments. For shorthand, unless explicitly stated otherwise, as used herein “neurological disease” contemplates at least ALS, DMD, SMA (such as SMA type I, SMA type II, or SMA type III), HD, AD, PD, MS, poliomyelitis (post-polio syndrome), spino-bulbar muscular atrophy (Kennedy syndrome), Guillain-Barré syndrome (GBS), chronic inflammatory demyelinated polyneuropathy (CIDP), critical illness polyneuropathy (CIP), and/or hereditary sensory neuropathies (HMSN), as well as subsets of two or more of any of listed items, and individual items in the list, for example, DMD, SMA (such as SMA type I, SMA type II, or SMA type III), HD, AD, PD, MS, poliomyelitis (post-polio syndrome), spino-bulbar muscular atrophy (Kennedy syndrome), GBS, CIDP, CIP, and/or HMS). For shorthand, unless explicitly stated otherwise, as used herein “neuromuscular disease” contemplates at least ALS, DMD, SMA (such as SMA type I, SMA type II, or SMA type III), and/or HD, as well as subsets of two or more of any of listed items, and individual items in the list, for example DMD, SMA (such as SMA type I, SMA type II, or SMA type III), and/or HD.

In the method (or corresponding use or composition for use) of some embodiments, the neurological disease comprises at least one of ALS, DMD, SMA (such as SMA type I, SMA type II, or SMA type III), HD, AD, PD, MS, poliomyelitis (post-polio syndrome), spino-bulbar muscular atrophy (Kennedy syndrome), GBS, CIDP, CIP, and/or HMSN. In the method (or corresponding use or composition for use) of some embodiments, the neurological disease comprises at least one of DMD, SMA (such as SMA type I, SMA type II, or SMA type III), HD, AD, PD, MS, poliomyelitis (post-polio syndrome), spino-bulbar muscular atrophy (Kennedy syndrome), GBS, CIDP, CIP, and/or HMSN. In the method (or corresponding use or composition for use) of some embodiments, the neurological disease comprises at least one of ALS, DMD, SMA (such as SMA type I, SMA type II, or SMA type III), Huntington's disease, poliomyelitis (post-polio syndrome), spino-bulbar muscular atrophy (Kennedy syndrome), GBS, CIDP, CUP, and/or HMSN. In the method (or corresponding use or composition for use) of some embodiments, the neurological disease comprises at least one of ALS, DMD, SMA (such as SMA type I, SMA type II, or SMA type III), Huntington's disease, poliomyelitis (post-polio syndrome), spino-bulbar muscular atrophy (Kennedy syndrome), GBS, CIDP, CIP, and/or HMSN. In the method (or corresponding use or composition for use) of some embodiments, the neurological disease comprises at least one of ALS, DMD, SMA (such as SMA type I, SMA type II, or SMA type III), Huntington's disease, poliomyelitis (post-polio syndrome), spino-bulbar muscular atrophy (Kennedy syndrome), GBS, CIDP, CIP, and/or HMSN. In the method (or corresponding use or composition for use) of some embodiments, the neurological disease comprises at least one of DMD, SMA (such as SMA type I, SMA type II, or SMA type III), Huntington's disease, poliomyelitis (post-polio syndrome), spino-bulbar muscular atrophy (Kennedy syndrome), GBS, CIDP, CIP, and/or HMSN. In the method (or corresponding use or composition for use) of some embodiments, the neurological disease comprises at least one of DMD, SMA (such as SMA type I, SMA type II, or SMA type III), poliomyelitis (post-polio syndrome), spino-bulbar muscular atrophy (Kennedy syndrome), GBS, CIDP, CIP, and/or HMSN. In the method (or corresponding use or composition for use) of some embodiments, the neurological disease comprises at least one of ALS, DMD, SMA (such as SMA type I, SMA type II, or SMA type III), poliomyelitis (post-polio syndrome), spino-bulbar muscular atrophy (Kennedy syndrome), GBS, CIDP, CIP, and/or HMSN. In the method (or corresponding use or composition for use) of some embodiments, the neurological disease comprises at least one of ALS, DMD, SMA (such as SMA type I, SMA type II, or SMA type III), HD, AD, PD and/or MS. In the method (or corresponding use or composition for use) of some embodiments, the neurological disease comprises at least one of DMD, SMA (such as SMA type L SMA type II, or SMA type III), HD, AD, PD and/or MS. In the method (or corresponding use or composition for use) of some embodiments, the neurological disease comprises at least one of ALS, HD, AD, PD, and/or MS. In the method (or corresponding use or composition for use) of some embodiments, the neurological disease comprises at least one of HD, AD, PD, and/or MS. In the method (or corresponding use or composition for use) of some embodiments, the neurological disease comprises at least one of ALS, AD, PD, and/or MS. In the method (or corresponding use or composition for use) of some embodiments, the neurological disease comprises at least one of AD, PD, and/or MS. In the method (or corresponding use or composition for use) of some embodiments, the neurological disease comprises at least one of ALS, DMD, SMA (such as SMA type I, SMA type II, or SMA type III), HD, and/or PD. In the method (or corresponding use or composition for use) of some embodiments, the neurological disease comprises at least one of ALS, DMD, SMA (such as SMA type I, SMA type II, or SMA type III), HD, and/or PD. In the method (or corresponding use or composition for use) of some embodiments, the neurological disease comprises at least one of ALS, DMD, SMA (such as SMA type I, SMA type II, or SMA type III), HD, and/or PD. In the method (or corresponding use or composition for use) of some embodiments, the neurological disease comprises at least one of ALS, DMD, SMA (such as SMA type I, SMA type II, or SMA type III), HD, and/or PD. In the method (or corresponding use or composition for use) of some embodiments, the neurological disease comprises at least one of ALS, DMD, SMA (such as SMA type I, SMA type II, or SMA type III), HD, and/or PD. In the method (or corresponding use or composition for use) of some embodiments, the neurological disease comprises at least one of ALS, DMD, SMA (such as SMA type I, SMA type II, or SMA type III), HD, and/or PD. In the method (or corresponding use or composition for use) of some embodiments, the neurological disease comprises at least one of ALS, DMD, SMA (such as SMA type I, SMA type II, or SMA type III), HD, and/or PD. In the method (or corresponding use or composition for use) of some embodiments, the neurological disease comprises at least one of ALS, DMD, SMA (such as SMA type I, SMA type II, or SMA type III), HD, and/or PD. In the method (or corresponding use or composition for use) of some embodiments, the neurological disease comprises at least one of ALS, DMD, SMA (such as SMA type I, SMA type II, or SMA type III), HD, and/or PD. In the method (or corresponding use or composition for use) of some embodiments, the neurological disease comprises at least one of ALS, DMD, SMA (such as SMA type I, SMA type II, or SMA type III), HD, and/or PD. In the method (or corresponding use or composition for use) of some embodiments, the neurological disease comprises at least one of DMD, SMA (such as SMA type I, SMA type II, or SMA type III), HD, and/or PD. In the method (or corresponding use or composition for use) of some embodiments, the neurological disease comprises at least one of ALS, DMD, SMA (such as SMA type I, SMA type II, or SMA type III), HD, PD and/or AD. In the method (or corresponding use or composition for use) of some embodiments, the neurological disease comprises at least one of ALS, DMD, SMA (such as SMA type I, SMA type II, or SMA type III), HD, PD and/or AD. In the method (or corresponding use or composition for use) of some embodiments, the neurological disease comprises at least one of ALS, DMD, SMA (such as SMA type I, SMA type II, or SMA type III), HD, PD and/or AD. In the method (or corresponding use or composition for use) of some embodiments, the neurological disease comprises at least one of ALS, DMD, SMA (such as SMA type I, SMA type II, or SMA type III), HD, PD and/or AD. In the method (or corresponding use or composition for use) of some embodiments, the neurological disease comprises at least one of ALS, DMD, SMA (such as SMA type I, SMA type II, or SMA type III), HD, PD and/or AD. In the method (or corresponding use or composition for use) of some embodiments, the neurological disease comprises at least one of ALS, DMD, SMA (such as SMA type I, SMA type II, or SMA type III), HD, PD and/or AD. In the method (or corresponding use or composition for use) of some embodiments, the neurological disease comprises at least one of DMD, SMA (such as SMA type I, SMA type II, or SMA type III), HD, PD and/or AD. In the method (or corresponding use or composition for use) of some embodiments, the neurological disease comprises at least one of ALS, DMD or SMA (such as SMA type I, SMA type II, or SMA type III). In the method (or corresponding use or composition for use) of some embodiments, the neurological disease comprises at least one of DMD or SMA (such as SMA type I, SMA type II, or SMA type III). In the method (or corresponding use or composition for use) of some embodiments, the neurological disease comprises at least one of ALS, DMD, SMA (such as SMA type I, SMA type II, or SMA type III), Huntington's disease, and/or AD. In the method (or corresponding use or composition for use) of some embodiments, the neurological disease comprises at least one of ALS, DMD, SMA (such as SMA type I, SMA type II, or SMA type III), Huntington's disease, and/or AD. In the method (or corresponding use or composition for use) of some embodiments, the neurological disease comprises at least one of DMD, SMA (such as SMA type I, SMA type II, or SMA type III), Huntington's disease, and/or AD. In the method (or corresponding use or composition for use) of some embodiments, the neurological disease comprises a neuromuscular disease such as at least one of ALS, DMD, SMA (such as SMA type I, SMA type II, or SMA type III), and/or HD. In the method (or corresponding use or composition for use) of some embodiments, the neurological disease comprises a neuromuscular disease such as at least one of DMD, SMA (such as SMA type I, SMA type II, or SMA type III), and/or HD. In the method (or corresponding use or composition for use) of some embodiments, the neurological disease comprises a neuromuscular disease such as at least one of ALS, DMD and/or HD. In the method (or corresponding use or composition for use) of some embodiments, the neurological disease comprises a neuromuscular disease such as at least one of DMD and/or HD. In the method (or corresponding use or composition for use) of some embodiments, the neurological disease comprises a neuromuscular disease such as at least one of ALS, DMD and/or SMA (such as SMA type I, SMA type II, or SMA type III). In the method (or corresponding use or composition for use) of some embodiments, the neurological disease comprises a neuromuscular disease such as at least one of DMD and/or SMA (such as SMA type I, SMA type II, or SMA type III). In the method (or corresponding use or composition for use) of some embodiments, the neurological disease comprises a neuromuscular disease such as at least one of ALS, SMA (such as SMA type I, SMA type II, or SMA type III), and/or HD. In the method (or corresponding use or composition for use) of some embodiments, the neurological disease comprises a neuromuscular disease such as at least one of SMA (such as SMA type I, SMA type II, or SMA type III), HD, and/or HD.

### Oxygenated Ionic Aqueous Solutions

“Oxygenated ionic aqueous solution,” as used herein, refers to solutions comprising, consisting essentially of; or consisting of electrokinetically-generated fluids comprising oxygen and ions. It will be understood that wherever “electrokinetically-generated fluids” (including variations of this root term) are mentioned herein, oxygenated ionic aqueous solutions comprising the electrokinetically-generated fluids are also expressly contemplated. In some embodiments, the oxygenated ionic aqueous solution comprises, consists essentially of; or consists of an ionic aqueous solution of charge-stabilized oxygen-containing nanostructures (such as nanobubbles), in which the majority of the nanostructures have an average diameter of less than about 100 nanometers and are stable in the ionic aqueous solution. The oxygenated ionic aqueous solutions are distinct from other oxygenated fluids, for example, oxygenated non-electrokinetic fluids (e.g., pressure pot oxygenated fluids and the like).

Oxygenated ionic aqueous solutions suitable for methods, medicaments, and uses of some embodiments herein can be produced, for example, using the Mixing Device described in detail in U.S. Pat. No. 9,745,567, which is herein incorporated by reference in its entirety. Methods and devices for making oxygenated ionic aqueous solutions are also described in detail in US Pub. No. 200802190088 and International Application No. WO2008/052143, each of which is herein incorporated by reference in its entirety. By way of example, suitable oxygenated ionic aqueous solutions for methods, medicaments, and uses of some embodiments herein can be generated in the presence of hydrodynamically-induced, localized (e.g., non-uniform with respect to the overall fluid volume) electrokinetic effects (e.g., voltage/current pulses), such as device feature-localized effects. In some embodiments, the oxygenated ionic aqueous solutions are characterized by hydrodynamically-induced, localized electrokinetic effects in combination with surface-related double layer and/or streaming current effects.

The oxygenated ionic aqueous solution of methods, uses, and medicaments of some embodiments comprises stabilized (e.g., charge-stabilized) oxygen-containing nanostructures. In some embodiments, the oxygenated ionic aqueous solution is superoxygenated, for example comprising at least 20 ppm, 40 ppm, or 60 ppm dissolved oxygen, in standard saline. It has been shown, for example, that ionic aqueous solutions having dissolved oxygen levels of 15 ppm or less can have physiological effects that are qualitatively similar to oxygenated ionic aqueous solutions having higher dissolved oxygen levels (See U.S. Pat. No. 9,745,567 at Examples 16 and 24), and oxygenated ionic aqueous solutions having oxygen levels of at least 20 ppm can inhibit cytokine markers of inflammation in vivo (See U.S. Pat. No. 9,745,567 at Example 13). Accordingly, in some embodiments, the oxygenated ionic aqueous solution comprises at least 15 ppm oxygen at standard temperature and pressure, such as at least 20 ppm, 25 ppm, 30 ppm, 35 ppm, 40 ppm, 45 ppm, 50 ppm, 55 ppm, 60 ppm, 65 ppm, 70 ppm, 75 ppm, or 80 ppm oxygen, including ranges between any two of the listed values, for example 15 ppm-80 ppm, 20 ppm-80 ppm, 25 ppm-80 ppm, 30 ppm-80 ppm, 35 ppm-80 ppm, 40 ppm-80 ppm, 45 ppm-80 ppm, 50 ppm-80 ppm, 55 ppm-80 ppm, 60 ppm-80 ppm, 15 ppm-75 ppm, 20 ppm-75 ppm, 25 ppm-75 ppm, 30 ppm-75 ppm, 35 ppm-75 ppm, 40 ppm-75 ppm, 45 ppm-75 ppm, 50 ppm-75 ppm, 55 ppm-75 ppm, 60 ppm-75 ppm, 15 ppm-70 ppm, 20 ppm-70 ppm, 25 ppm-70 ppm, 30 ppm-70 ppm, 35 ppm-70 ppm, 40 ppm-70 ppm, 45 ppm-70 ppm, 50 ppm-70 ppm, 55 ppm-70 ppm, 60 ppm-70 ppm, 15 ppm-65 ppm, 20 ppm-65 ppm, 25 ppm-65 ppm, 30 ppm-65 ppm, 35 ppm-65 ppm, 40 ppm-65 ppm, 45 ppm-65 ppm, 50 ppm-65 ppm, 55 ppm-65 ppm, 60 ppm-65 ppm, 15 ppm-60 ppm, 20 ppm-60 ppm, 25 ppm-60 ppm, 30 ppm-60 ppm, 35 ppm-60 ppm, 40 ppm-60 ppm, 45 ppm-60 ppm, 50 ppm-60 ppm, or 55 ppm-60 ppm of dissolved oxygen. In some embodiments, the oxygenated ionic aqueous solution comprises, consists essentially of, or consists of a saline solution. In some embodiments, the oxygenated ionic aqueous solution comprises, consists essentially of, or consists of a saline solution comprising at least 40 ppm oxygen. In some embodiments, the oxygenated ionic aqueous solution comprises stabilized (e.g., charge-stabilized) oxygen-containing nanostructures in an amount sufficient to provide modulation of at least one of cellular membrane potential and cellular membrane conductivity.

In some embodiments, the dissolved oxygen content, salinity, sterility, pH, etc., of the oxygenated ionic aqueous solution is established at the time of electrokinetic production of the fluid. As shown in Example 1, dissolved oxygen levels of oxygenated ionic aqueous solutions in accordance with some embodiments herein can remain stable in a sealed container for many months. Accordingly, it is contemplated that a dissolved oxygen content of an oxygenated ionic aqueous solution, for example as a pharmaceutical product, at the time it was manufactured can be a suitable way of identifying the oxygenated ionic aqueous solution (as it may be impractical to determine a dissolved oxygen content at the exact time of clinical use). For example, the oxygenated ionic aqueous solution of methods (or corresponding uses or medicaments) some embodiments can have had a specified level of dissolved oxygen at the time it was manufactured, for example at least about 20 ppm dissolved oxygen. The amount of dissolved oxygen can refer to an amount at standard temperature and pressure, though this is to simply in reference to a way of making a measurement, and in no way should be constructed to require that any or all of the manufacturing be performed at standard temperature and/or pressure. In some embodiments, the oxygenated ionic aqueous solution has a dissolved oxygen content (at standard temperature and pressure) of at least 15 ppm dissolved oxygen (at standard temperature and pressure, such as at least 20 ppm, 25 ppm, 30 ppm, 35 ppm, 40 ppm, 45 ppm, 50 ppm, 55 ppm, 60 ppm, 65 ppm, 70 ppm, 75 ppm, or 80 ppm, including ranges between any two of the listed values, for example, 20 ppm-80 ppm, 25 ppm-80 ppm, 30 ppm-80 ppm, 35 ppm-80 ppm, 40 ppm-80 ppm, 45 ppm-80 ppm, 50 ppm-80 ppm, 55 ppm-80 ppm, 60 ppm-80 ppm, 15 ppm-75 ppm, 20 ppm-75 ppm, 25 ppm-75 ppm, 30 ppm-75 ppm, 35 ppm-75 ppm, 40 ppm-75 ppm, 45 ppm-75 ppm, 50 ppm-75 ppm, 55 ppm-75 ppm, 60 ppm-75 ppm, 15 ppm-70 ppm, 20 ppm-70 ppm, 25 ppm-70 ppm, 30 ppm-70 ppm, 35 ppm-70 ppm, 40 ppm-70 ppm, 45 ppm-70 ppm, 50 ppm-70 ppm, 55 ppm-70 ppm, 60 ppm-70 ppm, 15 ppm-65 ppm, 20 ppm-65 ppm, 25 ppm-65 ppm, 30 ppm-65 ppm, 35 ppm-65 ppm, 40 ppm-65 ppm, 45 ppm-65 ppm, 50 ppm-65 ppm, 55 ppm-65 ppm, 60 ppm-65 ppm, 15 ppm-60 ppm, 20 ppm-60 ppm, 25 ppm-60 ppm, 30 ppm-60 ppm, 35 ppm-60 ppm, 40 ppm-60 ppm, 45 ppm-60 ppm, 50 ppm-60 ppm, or 55 ppm-60 ppm of dissolved oxygen at the time that the oxygenated ionic aqueous solution was manufactured. In some embodiments, the oxygenated ionic aqueous solution is a pharmaceutical saline solution that has one of the above-noted dissolved oxygen contents at the time that the oxygenated ionic aqueous solution was manufactured. In some embodiments, the oxygenated ionic aqueous solution is a saline solution comprising at least 40 ppm oxygen. In some embodiments, any of the oxygenated ionic aqueous solutions described herein comprises stabilized oxygen-containing nanostructures (such as nanobubbles), a majority of the nanostructures having a diameter of less than 100 nanometers.

Oxygenated ionic aqueous solutions of methods, uses, and medicaments of some embodiments comprise modified or charged oxygen species. In some embodiments, the oxygen of the oxygenated ionic aqueous solution comprises, consists essentially of, or consists of molecular oxygen. In some embodiments, the oxygenated ionic aqueous solution is free of ozone, or comprises no more than trace amounts of ozone (e.g., amounts of ozone that have no observable physical or physiological effect). Without being limited by theory, it is contemplated that the oxygenated ionic aqueous solutions of methods, uses, and medicaments of some embodiments comprises at least one of a form of solvated electrons, and electrokinetically modified or charged oxygen species. The electrokinetic modification can comprise, consist essentially of, or consist of oxygen-containing nanostructures stabilized by an imparted charge. In some embodiments, the oxygenated ionic aqueous solution comprises solvated electrons stabilized by molecular oxygen. In some embodiments, the solvated electrons or electrokinetically modified or charged oxygen species are present in the oxygenated ionic aqueous solution in an amount of at least 0.01 ppm, at least 0,1 ppm, at least 0.5 ppm, at least 1 ppm, at least 3 ppm, at least 5 ppm, at least 7 ppm, at least 10 ppm, at least 15 ppm, or at least 20 ppm. In some embodiments, the oxygenated ionic aqueous solution comprises solvated electrons stabilized, at least in part, by molecular oxygen.

It is noted that the oxygenated ionic aqueous solutions in accordance with methods, uses, and medicaments of some embodiments have been shown to be stable in sealed containers for long periods of time (See, e.g., Example 1). In some embodiments, for the oxygenated ionic aqueous solution of any of the methods, uses, or medicaments herein, oxygen in the oxygenated ionic aqueous solution has been present in an amount of at least 15 ppm at standard temperature and pressure for at least 3 hours. In some embodiments, for the oxygenated ionic aqueous solution of any of the methods, uses, or medicaments herein, oxygen in the oxygenated ionic aqueous solution has been present in an amount of at least 15 ppm at standard temperature and pressure for at least 1 month, such as at least 2, 3, 4, 5, or 6 months. In some embodiments, for the oxygenated ionic aqueous solution of any of the methods, uses, or medicaments herein, oxygen in the oxygenated ionic aqueous solution has been present in an amount of at least 40 ppm at standard temperature and pressure for at least 3 hours. In some embodiments, for the oxygenated ionic aqueous solution of any of the methods, uses, or medicaments herein, oxygen in the oxygenated ionic aqueous solution has been present in an amount of at least 40 ppm at standard temperature and pressure for at least 1 month, such as at least 2, 3, 4, 5, or 6 months.

Oxygenated ionic aqueous solutions can be sterile and can be administered by an appropriate route. In some embodiments, at least one of the salinity, sterility, pH, etc., of the oxygenated ionic aqueous solution is appropriately adjusted (e.g., using sterile saline or appropriate diluents) to be physiologically compatible with the route of administration prior to administration of the fluid. Preferably, and diluents and/or saline solutions and/or buffer compositions used to adjust at least one of the salinity, sterility, pH, etc., of the fluids are also electrokinetic fluids, or are otherwise compatible.

In some embodiments, the oxygenated ionic aqueous solution comprises saline (e.g., one or more dissolved salt(s); e.g., alkali metal based salts (Li⁺, Na⁺, K⁺, Rb⁺, Cs⁺, etc.), alkaline earth based salts (e.g., Mg⁺⁺, Ca⁺⁺), etc., or transition metal-based positive ions (e.g., Cr, Fe, Co, Ni, Cu, Zn, etc.,), in each case along with any suitable anion components, including, but not limited to F⁻, Cl⁻, Br⁻, I⁻, PO4⁻, SO4⁻, and nitrogen-based anions. Particular aspects comprise mixed salt based ionic solutions (e.g., Na⁺, K⁺, Ca⁺⁺, Mg⁺⁺, transition metal ion(s), etc.) in various combinations and concentrations, and optionally with mixtures of counterions. In some embodiments, the oxygenated ionic aqueous solution comprises standard saline (e.g., approx. 0.9% NaCl, or about 0.15 M NaCl). In particular aspects, the oxygenated ionic aqueous solution of methods, uses, and medicaments of some embodiments comprises saline at a concentration of at least 0.0002 M, at least 0.0003 M, at least 0.001 M, at least 0.005 M, at least 0.01 M, at least 0.015 M, at least 0.1 M, at least 0.15 M, or at least 0.2 M. In some embodiments, the conductivity of the oxygenated ionic aqueous solution is at least 10 μS/cm, at least 40 μS/cm, at least 80 μS/cm, at least 100 μS/cm, at least 150 μS/cm, at least 200 μS/cm, at least 300 μS/cm, or at least 500 μS/cm, at least 1 mS/cm, at least 5, mS/cm, 10 mS/cm, at least 40 mS/cm, at least 80 mS/cm, at least 100 mS/cm, at least 150 mS/cm, at least 200 mS/cm, at least 300 mS/cm, or at least 500 mS/cm. In some embodiments, any salt may be comprised by the oxygenated ionic aqueous solution, provided that they allow for formation of biologically active salt-stabilized nanostructures (e.g., salt-stabilized oxygen-containing nanostructures) as disclosed herein.

The oxygenated ionic aqueous solution of some embodiments can be for use as a medicament, or for medical use. In some embodiments, the oxygenated ionic aqueous solution is part of a pharmaceutical composition. As such, wherever a method, use, or medicament comprising the oxygenated ionic aqueous solution is mentioned herein, the corresponding method, use, or medicament comprising a pharmaceutical composition comprising the oxygenated ionic aqueous solution is also expressly contemplated. The pharmaceutical composition can comprise, consist essentially of, or consist of the oxygenated ionic aqueous solution. In some embodiments, the pharmaceutical composition comprises an active ingredient in addition to the oxygenated ionic aqueous solution. In some embodiments, the oxygenated ionic aqueous is formulated for intravenous administration. In some embodiments, the oxygenated ionic aqueous is formulated for inhalation, such as spray or nebulization. In some embodiments, the oxygenated ionic aqueous solution is administered to the patient in a sterile composition, which can comprise, consists essentially of, or consist of the oxygenated ionic aqueous solution. As such, the oxygenated ionic aqueous solution can be administered to the patient in a composition that does not comprise tissues or cells, for example a composition that does not comprise blood. In some embodiments, the oxygenated ionic aqueous solution is administered to the patient in a solution that does not comprise blood.

In some embodiments, the oxygenated ionic aqueous solution has anti-inflammatory and/or neuroprotective effects in a patient in need thereof, for example DMD, SMA (type I, II, and/or III), HD, AD, and/or PD. For example, in some embodiments, the oxygenated ionic aqueous solution is effective in reduction of peripheral inflammation (effector T cells down and/or regulatory T cells up), reduction of CNS inflammation (decrease in inflammatory glia and/or reduction of NFkB activity), increase in non-inflammatory microglia, increase in survival, maturation and function of oligodendrocytes, augmentation of neuronal branching, plasticity and neurotransmission, enhancement of neuronal survival (anti-apoptotic), and/or stimulation of mitochondrial biogenesis and function. For example, oxygenated ionic aqueous solution in accordance with some embodiments has been shown to ameliorate impaired mitochondrial respiration (Example 9). Accordingly, in the method of some embodiments, the patient is selected as having a deficiency in mitochondrial function, for example impaired mitochondrial respiration. Without being limited by theory, the oxygenated ionic aqueous solution is contemplated to be effective to exert neuroprotective effects in neurological diseases, for example on motor neurons in neuromuscular diseases that comprise a decline in respiratory function, thus inhibiting, reducing, or slowing the decline in respiratory function.

Methods of Ameliorating, Inhibiting, Reducing, Treating, Slowing the Progression of, or Preventing a Disease Decline in Respiratory Function Associated with a Neurological Disease of a Patient

Some embodiments include a method of ameliorating, inhibiting, reducing, treating, slowing the progression of, or preventing a decline in respiratory function associated with a neurological disease of a patient. The method can comprise selecting the patient. By way of example, the patient can be selected as having the neurological disease and undergoing a decline in respiratory function, having the neurological disease and at risk of a decline in respiratory function, at risk of the neurological disease and undergoing a decline in respiratory function, or at risk of the neurological disease and at risk of undergoing a decline in respiratory function. The method can comprise administering an effective amount of oxygenated ionic aqueous solution to the patient, thus ameliorating, inhibiting, reducing, treating, slowing the progression of, or preventing the decline in respiratory function associated with the neurological disease. The decline in respiratory function can comprise, consist essentially of, or consist of a decline in vital capacity such as SVC and/or FVC. It will be appreciated that wherever a method of ameliorating, inhibiting, reducing, treating, slowing the progression of, or preventing a decline in respiratory function associated with a neurological disease comprising administering an oxygenated ionic aqueous solution is mentioned, an oxygenated ionic aqueous solution (or pharmaceutical composition comprising the oxygenated ionic aqueous solution) for use in treating, slowing the progression of, or preventing the decline in respiratory function associated with a neurological disease is expressly contemplated as well.

In some embodiments, the oxygenated ionic aqueous solution comprises at least 20 ppm oxygen at the time it was manufactured (such as at least 30 ppm, 40 ppm, 50 ppm, 60 ppm, 70 ppm, or 80 ppm). By way of example, the oxygenated ionic aqueous solution of some embodiments of the method of inhibiting, treating, preventing, ameliorating, or reducing a symptom of ALS as described herein can be a pharmaceutical saline solution comprising stabilized oxygen-containing nanostructures, a majority of the nanostructures having a diameter of less than 100 nanometers. The pharmaceutical saline solution can comprise greater than or equal to 50 ppm oxygen at the time that the pharmaceutical saline solution was manufactured.

It has been shown herein that treatment of neurological disease patients with the oxygenated ionic aqueous solution in accordance with some embodiments can inhibit the rate of decline of slow vital capacity (SVC), so that the decline in SVC is slower, or there is no decline. See Example 2. In particular, treatment of ALS patients with oxygenated ionic aqueous solution in accordance with some embodiments has been shown to slow the rate of decrease of SVC by about 5-fold in ALS patients. Remarkably, 5 out of 11 patients showed no decline in SVC (Example 2). Moreover, since the oxygenated ionic aqueous solution has been shown to have neuroprotective effects (Examples 3-8), it is contemplated that the oxygenated ionic aqueous solution of some embodiments can be useful in patients having (or at risk of having) neurological disease as described herein, which may also be associated with decline and/or damage to motor neurons, glia, and/or neuromuscular junctions for example, at least one of DMD, SMA (type I, type II, and/or type II), Huntington's disease, AD, and/or PD. In some embodiments, the patient has at least one of DMD and/or SMA. In some embodiments, the patient does not have ALS.

In some embodiments, the oxygenated ionic aqueous solution is administered to the subject repeatedly over a period of weeks. The administering can comprise intravenous administration, inhalation, or intravenous administration and inhalation as described herein. In the method of some embodiments, the period of weeks over which the oxygenated ionic aqueous solution is administered comprises at least 4 weeks, for example as at least about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 weeks, including ranges between any two of the listed values. In some embodiments, the period of weeks is at least about 20 weeks. In some embodiments, the period of weeks is at least about 23 weeks. In some embodiments, the period of weeks is about 4-20 weeks, 4-30 weeks, 4-40 weeks, 4-50 weeks, 4-60 weeks, 6-20 weeks, 6-30 weeks, 6-40 weeks, 6-50 weeks, 6-60 weeks, 8-20 weeks, 8-30 weeks, 8-40 weeks, 8-50 weeks, 8-60 weeks, 10-20 weeks, 10-30 weeks, 10-40 weeks, 10-50 weeks, 10-60 weeks, 20-30 weeks, 20-40 weeks, 20-50 weeks, 20-60 weeks, 30-40 weeks, 30-50 weeks, 30-60 weeks, 40-50 weeks, 40-60 weeks, or 50-60 weeks.

In some embodiments, the method further comprises ameliorating, inhibiting, reducing, treating, slowing the progression of, or preventing a decline of respiratory function (or the rate of decline of respiratory function). In some embodiments, ameliorating, inhibiting, reducing, treating, slowing the progression of, or preventing the decline of respiratory function (or the rate of decline of respiratory function) in the patient comprises reducing the rate at which vital capacity (e.g., SVC and/or FVC) declines, halting a decline in vital capacity, or increasing vital capacity. In some embodiments, ameliorating, inhibiting, reducing, treating, slowing the progression of, or preventing the decline of respiratory function (or the rate of decline of respiratory function) in the patient comprises reducing the rate at which vital capacity declines, or halting a decline in vital capacity. In some embodiments, ameliorating, inhibiting, reducing, treating, slowing the progression of, or preventing the decline of respiratory function (or the rate of decline of respiratory function) in the patient comprises reducing the rate at Which vital capacity declines. In some embodiments, ameliorating, inhibiting, reducing, treating, slowing the progression of, or preventing the decline of respiratory function (or the rate of decline of respiratory function) comprises at least a 10%, 20%, 30%, 40%, 50%, 60%, 70%, 80%, 90%, 95%, 99%, or 100% including ranges between any two of the listed values, for example a 10%400% decrease, 10%-20%, 10%-30%, 10%-40%, 10%-50%, 10%-60%, 10%-70%, 10%-80%, 10%-90%, 10%-95%, 10%-99%, or 10%-100% decrease in decline of vital capacity, and/or absence of decline in vital capacity.

In the method of some embodiments, the ameliorating, inhibiting, reducing, treating, slowing the progression of, or preventing the decline of respiratory function (or the rate of decline of respiratory function) in the patient is detected. In some embodiments, the method of comprises detecting an inhibition, reduction, treatment, a slowing in the progression of, or prevention of inhibition of decline in vital capacity (e.g, SVC and/or FVC) following the administration of the oxygenated ionic aqueous solution. In some embodiments, the method further comprises detecting at least a 10%, 20%, 30%, 40%, 50%, 60%, 70%, 80%, 90%, 95%, 99%, or 100% including ranges between any two of the listed values, for example a 10%-100% decrease, 10%-20%, 10%-30%, 10%-40%, 10%-50%, 10%-60%, 10%-70%, 10%-80%, 10%-90%, 10%-95%, 10%-99%, 10%-100%, 10%-30%, 10%-40%, 10%-50%, 10%-60%, 10%-70%, 10%-80%, 10%-90%, 10%-95%, 10%-99%, or 10%-100% decrease in decline of vital decrease, and/or absence of decline in vital capacity (e.g., SVC and/or FVC) (it is noted that an absence of vital capacity decline as described herein would also be considered a decrease in the rate of vital capacity decline). In some embodiments, the method (or corresponding use or medicament) further comprises detecting an absence of decline in vital capacity (e.g., SVC and/or FVC) following the administration of the oxygenated ionic aqueous solution.

It is contemplated that interventions for a decline in respiratory function, such as ventilators, can carry their own risks, or have undesirable side effects. Accordingly, it can be advantageous to defer ventilator treatment through treatment with an oxygenated aqueous ionic solution in accordance with some embodiments herein. Thus, deferring a ventilator by treatment with oxygenated aqueous ionic solutions in some embodiments can defer or avoid such risks and side effects. Moreover, treatment of patients having the neurological disease and undergoing a decline in respiratory function with oxygenated aqueous ionic solution some embodiments can increase survival of the patient. The survival can be increased beyond a time that would have been expected if the patient had not received the oxygenated aqueous ionic solution. In some embodiments, in the absence of administering the oxygenated aqueous ionic solutions, the ventilator would have been expected at a time, and the ventilator is deferred beyond the expected time.

In any of the methods described herein, a ventilator can be deferred at least 1 week, such as at least 2 weeks, 3 weeks, 4 weeks, 5 weeks, 6 weeks, 7 weeks, 8 weeks, 9 weeks, 10 weeks, 11 weeks, or 12 weeks, including ranges between any two of the listed values, such as 1-12 weeks, such as 1-2 weeks, 1-3 weeks, 1-4 weeks, 1-5 weeks, 1-6 weeks, 1-7 weeks, 1-8 weeks, 1-9 weeks, 1-10 weeks, 1-11 weeks, 1-12 weeks, 2-3 weeks, 2-4 weeks, 2-5 weeks, 2-6 weeks, 2-7 weeks, 2-8 weeks, 2-9 weeks, 2-10 weeks, 2-11 weeks, 2-12 weeks, 3-4 weeks, 3-5 weeks, 3-6 weeks, 3-7 weeks, 3-8 weeks, 3-9 weeks, 3-10 weeks, 3-11 weeks, 3-12 weeks, 4-5 weeks, 4-6 weeks, 4-7 weeks, 4-8 weeks, 4-9 weeks, 4-10 weeks, 4-11 weeks, 4-12 weeks, 5-6 weeks, 5-7 weeks, 5-8 weeks, 5-9 weeks, 5-10 weeks, 5-11 weeks, 5-12 weeks, 6-7 weeks, 6-8 weeks, 6-9 weeks, 6-10 weeks, 6-11 weeks, or 6-12 weeks. It is noted that perpetually or continually deferring a ventilator (e.g., because a patient does not suffer a decline in vital capacity due to treatment with an oxygenated ionic aqueous fluid of some embodiments) are also contemplated as “deferring” the ventilator in accordance with the methods, uses, and medicaments of embodiments herein. in some embodiments, the ventilatory is perpetually or continually deferred.

For any of the methods described herein, in some embodiments, the decline in respiratory function comprises, consists essentially of, or consists of a decline in vital capacity. In some embodiments, any of the methods described herein further comprises detecting an inhibition or reduction or decline in vital capacity (for example SVC and/or FVC) in the patient following said administration.

For any of the methods described herein, in some embodiments, ameliorating, inhibiting, reducing, treating, slowing the progression of, or preventing the decline of respiratory function (or the rate of decline of respiratory function) comprises reducing a rate at which vital capacity declines, halting a decline in vital capacity, or increasing vital capacity. For any of the methods described herein, in some embodiments, ameliorating, inhibiting, reducing, treating, slowing the progression of, or preventing the decline of respiratory function (or the rate of decline of respiratory function) comprises reducing a rate at which vital capacity declines or halting a decline in vital capacity. For any of the methods described herein, in some embodiments, the vital capacity comprises SVC and/or FVC.

It is contemplated that for any of the methods described herein, a patient can be selected as likely to benefit from oxygenated ionic aqueous solution, for example by having, or being at risk of having a neurological disease as described herein and/or a decline in respiratory function. In some embodiments, the patient is selected as having the neurological disease and undergoing the decline in respiratory function or having the neurological disease and being at risk of the decline in respiratory function. In some embodiments, the patient is selected as being at risk of the neurological disease and being at risk of the decline in respiratory function, or being at risk of the neurological disease and undergoing the decline in respiratory function. In some embodiments, the patient is selected as being at risk of the neurological disease and undergoing the decline in respiratory function, or having the neurological disease and undergoing the decline in respiratory function. In some embodiments, the patient is selected as being at risk of the neurological disease and being at risk of the decline in respiratory function, or having the neurological disease and being at risk of the decline in respiratory function.

### Methods of Inhibiting, Treating, Preventing, Ameliorating, or Reducing a Symptom of ALS, and Methods of Deferring an ALS Intervention in an ALS Patient

Some embodiments include a method of inhibiting, treating, preventing, ameliorating, or reducing a symptom of ALS. The method can comprise administering an oxygenated ionic aqueous solution to an ALS patient repeatedly over a period of weeks. The administering can comprise intravenous administration, inhalation, or intravenous administration and inhalation. The method can comprise deferring an ALS intervention in the patient after the oxygenated ionic aqueous solution is administered. Example ALS interventions that can be deferred include use of a ventilator and/or a tracheotomy. In some embodiments, the ALS intervention is deferred from a future time at which the patient would be expected to need the intervention in the absence of treatment with the oxygenated ionic aqueous solution. The future point in time can be at or after the time at which administration of the oxygenated ionic aqueous solution begins. That is, in some embodiments, in the absence of administering the oxygenated ionic aqueous solution, the ALS intervention would have been expected at a time, and upon administration of the oxygenated ionic aqueous solution, the ALS intervention is deferred beyond the expected time. In some embodiments, the expected time of an ALS intervention is estimated based on an expected rate of vital capacity (e.g., SVC and/or FVC) decline (in the absence of treatment with the oxygenated ionic aqueous solution), and the ALS intervention is deferred from a time calculated according to the expected rate of vital capacity (e.g., SVC and/or FVC) decline (in the absence of treatment with the oxygenated ionic aqueous solution). In some embodiments, the oxygenated ionic aqueous solution comprised at least 20 ppm oxygen at the time it was manufactured (such as at least 30 ppm, 40 ppm, 50 ppm, 60 ppm, 70 ppm, or 80 ppm).

By way of example, the oxygenated ionic aqueous solution of some embodiments of the method of inhibiting, treating, preventing, ameliorating, or reducing a symptom of ALS as described herein can be a pharmaceutical saline solution comprising stabilized oxygen-containing nanostructures (e.g., nanobubbles), a majority of the nanostructures (e.g., nanobubbles)having a diameter of less than 100 nanometers. The pharmaceutical saline solution can comprise greater than or equal to 50 ppm oxygen at the time that the pharmaceutical saline solution was manufactured.

Some embodiments include a method of deferring an ALS intervention in an ALS patient. The method can comprise identifying the ALS patient as in need of the ALS intervention at a future time if the patient is untreated with an oxygenated ionic aqueous solution. The method can further include administering the oxygenated ionic aqueous solution to the ALS patient repeatedly over a period of weeks. By way of example, the administering can comprise intravenous administration, inhalation, or intravenous administration and inhalation. The method can comprise deferring the ALS intervention beyond the future time by at least 1 week. In some embodiments, the oxygenated ionic aqueous solution comprised at least 20 ppm oxygen at the time it was manufactured (such as at least 30 ppm, 40 ppm, 50 ppm, 60 ppm, 70 ppm, or 80 ppm). By way of example, the oxygenated ionic aqueous solution of some embodiments of the method of deferring an ALS intervention as described herein can be a pharmaceutical saline solution comprising stabilized oxygen-containing nanostructures (e.g., nanobubbles), a majority of the nanostructures (e.g., nanobubbles) having a diameter of less than 100 nanometers. The pharmaceutical saline solution can comprise greater than or equal to 50 ppm oxygen at the time that the pharmaceutical saline solution was manufactured.

It has been shown herein that treatment of ALS patients with the oxygenated ionic aqueous solution in accordance with some embodiments can inhibit the rate of decline of slow vital capacity (SVC), so that the decline in SVC is slower, or there is no decline. See Example 2. In particular, treatment of ALS patients with oxygenated ionic aqueous solution in accordance with some embodiments has been shown to slow the rate of decrease of SVC by about 5-fold in ALS patients. Remarkably, 5 out of 11 patients showed no decline in SVC (Example 2). Accordingly, in some embodiments, the method of inhibiting, treating, preventing, ameliorating, or reducing the symptoms of ALS, or of deferring an ALS intervention (or in the corresponding use or medicament) further comprises ameliorating, inhibiting, reducing, treating, slowing the progression of, or preventing a decline of respiratory function (or the rate of decline of respiratory function) in the ALS patient. The patient can suffer from a decline in respiratory function, or be at risk of a decline in respiratory function. The decline in respiratory function can comprise, consist essentially of, or consist of a decline in vital capacity such as SVC and/or FVC. In some embodiments, the method comprises ameliorating, inhibiting, reducing, treating, slowing the progression of, or preventing the decline of respiratory function (or the rate of decline of respiratory function) in the patient comprises reducing the rate at which vital capacity declines, halting a decline in vital capacity, or increasing vital capacity. In some embodiments, the method comprises ameliorating, inhibiting, reducing, treating, slowing the progression of, or preventing the decline of respiratory function (or the rate of decline of respiratory function) in the patient comprises reducing the rate at which vital capacity declines, or halting a decline in vital capacity. In some embodiments, the method comprises ameliorating, inhibiting, reducing, treating, slowing the progression of, or preventing the decline of respiratory function (or the rate of decline of respiratory function) in the patient comprises reducing the rate at which vital capacity declines. In some embodiments, ameliorating, inhibiting, reducing, treating, slowing the progression of, or preventing the decline of respiratory function (or the rate of decline of respiratory function) comprises at least a 10% decrease in the rate of decline in vital capacity (e.g., SVC and/or FVC) following the administration of the oxygenated ionic aqueous solution, for example at least a 10%, 20%, 30%, 40%, 50%, 60%, 70%, 80%, 90%, 95%, 99%, or 100% including ranges between any two of the listed values, for example a 10%-100% decrease, 10%-20%, 10%-30%, 10%-40%, 10%-50%, 10%-60%, 10%-70%, 10%-80%, 10%-90%, 10%-95%, 10%-99%, 10%-100%, 10%-30%, 10%-40%, 10%-50%, 10%-60%, 10%-70%, 10%-80%, 10%-90%, 10%-95%, 10%-99%, or 10%-100% decrease in decline in vital capacity and/or absence of decline in vital capacity.

In some embodiments, ameliorating, inhibiting, reducing, treating, slowing the progression of, or preventing the decline of respiratory function (or the rate of decline of respiratory function) in the patient is detected. Accordingly, in some embodiments, the method of inhibiting, treating, preventing, ameliorating, or reducing the symptoms of ALS, or of deferring an ALS intervention (or in the corresponding use or medicament) further comprises detecting an inhibition of decline in vital capacity (e.g, SVC and/or FVC) following the administration of the oxygenated ionic aqueous solution. Accordingly, in some embodiments, the method (or corresponding use or medicament) further comprises detecting at least a 10% decrease in the rate of decline in vital capacity (e.g., SVC and/or FVC) following the administration of the oxygenated ionic aqueous solution, for example at least a 10%, 20%, 30%, 40%, 50%, 60%, 70%, 80%, 90%, 95%, 99%, or 100% including ranges between any two of the listed values, for example a 10%-100% decrease, 10%-20%, 10%-30%, 10%-40%, 10%-50%, 10%-60%, 10%-70%, 10%-80%, 10%-90%, 10%-95%, 10%-99%, 10%-100%, 10%-30%, 10%-40%, 10%-50%, 10%-60%, 10%-70%, 10%-80%, 10%-90%, 10%-95%, 10%-99%, or 10%400% decrease in decline in vital capacity and/or absence of decline in vital capacity (e.g., SVC and/or FVC) (it is noted that an absence of vital capacity decline as described herein would also be considered a decrease in the rate of vital capacity decline). In some embodiments, the method (or corresponding use or medicament) further comprises detecting an absence of decline in vital capacity (e.g., SVC and/or FVC) following the administration of the oxygenated ionic aqueous solution.

It is contemplated that ALS interventions can carry their own risks, or have undesirable side effects. Accordingly, it can be advantageous to defer ALS interventions through treatment with an oxygenated aqueous ionic solution in accordance with some embodiments herein. Examples of ALS interventions that can be deferred in methods of inhibiting, treating, preventing, ameliorating, or reducing a symptom of ALS, or of deferring an ALS intervention in an ALS patient as described herein (or the corresponding uses or medicaments) include ventilator and/or tracheotomy. it is noted that either a ventilator or tracheotomy can cause discomfort, and an elevated risk of infection (such as ventilator-associated pneumonia). Thus, deferring ALS interventions by treatment with oxygenated aqueous ionic solution some embodiments can defer or avoid such risks and side effects. Moreover, treatment of ALS patients with oxygenated aqueous ionic solution some embodiments can increase survival of the patient. The survival can be increased beyond a time that would have been expected if the patient had not received the oxygenated aqueous ionic solution.

In any of the methods of inhibiting, treating, preventing, ameliorating, or reducing the symptoms of ALS, or of deferring an ALS intervention in an ALS patient as described herein (or the corresponding use or medicament), the ALS intervention can be deferred at least 1 week, such as at least 2 weeks, 3 weeks, 4 weeks, 5 weeks, 6 weeks, 7 weeks, 8 weeks, 9 weeks, 10 weeks, 11 weeks, or 12 weeks, including ranges between any two of the listed values, such as 1-12 weeks, such as 1-2 weeks, 1-3 weeks, 1-4 weeks, 1-5 weeks, 1-6 weeks, 1-7 weeks, 1-8 weeks, 1-9 weeks, 1-10 weeks, 1-11 weeks, 1-12 weeks, 2-3 weeks, 2-4 weeks, 2-5 weeks, 2-6 weeks, 2-7 weeks, 2-8 weeks, 2-9 weeks, 2-10 weeks, 2-11 weeks, 2-12 weeks, 3-4 weeks, 3-5 weeks, 3-6 weeks, 3-7 weeks, 3-8 weeks, 3-9 weeks, 3-10 weeks, 3-11 weeks, 3-12 weeks, 4-5 weeks, 4-6 weeks, 4-7 weeks, 4-8 weeks, 4-9 weeks, 4-10 weeks, 4-11 weeks, 4-12 weeks, 5-6 weeks, 5-7 weeks, 5-8 weeks, 5-9 weeks, 5-10 weeks, 5-11 weeks, 5-12 weeks, 6-7 weeks, 6-8 weeks, 6-9 weeks, 6-10 weeks, 6-11 weeks, or 6-12 weeks. It is noted that perpetually or continually deferring ALS interventions (e.g., because an ALS patient does not suffer a decline in vital capacity due to treatment with an oxygenated ionic aqueous fluid of some embodiments) are also contemplated as “deferring” the ALS intervention in accordance with the methods, uses, and medicaments of embodiments herein. In some embodiments, the ALS intervention is perpetually or continually deferred.

In the method of some embodiments, the period of weeks over which the oxygenated ionic aqueous solution is administered comprises at least 4 weeks, for example as at least about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 weeks, including ranges between any two of the listed values. In some embodiments, the period of weeks is at least about 20 weeks. In some embodiments, the period of weeks is at least about 23 weeks. In some embodiments, the period of weeks is about 4-20 weeks, 4-30 weeks, 4-40 weeks, 4-50 weeks, 4-60 weeks, 6-20 weeks, 6-30 weeks, 6-40 weeks, 6-50 weeks, 6-60 weeks, 8-20 weeks, 8-30 weeks, 8-40 weeks, 8-50 weeks, 8-60 weeks, 10-20 weeks, 10-30 weeks, 10-40 weeks, 10-50 weeks, 10-60 weeks, 20-30 weeks, 20-40 weeks, 20-50 weeks, 20-60 weeks, 30-40 weeks, 30-50 weeks, 30-60 weeks, 40-50 weeks, 40-60 weeks, or 50-60 weeks.

### Methods of Ameliorating, Inhibiting, Reducing, Treating, Slowing the Progression of, or Preventing a Decline in Respiratory Function (Or the Rate of Decline of Respiratory Function) in a Patient

It has been shown herein that oxygenated ionic aqueous solution in accordance with some embodiments can inhibit the rate of decline of slow vital capacity (SVC), so that the decline in SVC is slower, or there is no decline. See Example 2. Accordingly, it is contemplated that oxygenated ionic aqueous solutions in accordance with some embodiments herein can be used to inhibit, reduce, treat, slow the progression of, or prevent a decline in respiratory function (or the rate of decline of respiratory function) in a patient in need of the same. In some embodiments, a method of ameliorating, inhibiting, reducing, treating, slowing the progression of, or preventing a decline in respiratory function (or the rate of decline of respiratory function) in a patient is described. The method can comprise selecting the patient as undergoing a decline in respiratory function, or at risk of undergoing a decline in respiratory function. The method can comprise administering an oxygenated ionic aqueous solution to the patient as described herein. The oxygenated ionic aqueous solution can be administered repeatedly over a period of weeks, as described herein. The administering can comprise intravenous administration, inhalation, or intravenous administration and inhalation. By way of example, the patient can suffer from a neurological disease as described herein, which can cause a decline in respiratory function, or a risk of undergoing a decline in respiratory function.

As administration of the oxygenated ionic aqueous solution as described herein can ameliorate, inhibit, reduce, treat, slow the progression of, or prevent a decline in respiratory function (or the rate of decline of respiratory function), it is contemplated that the following administration of the oxygenated ionic aqueous solution, a treatment for impaired respiratory function can be deferred or avoided. In some embodiments, the method comprises deferring use of a ventilator for the patient for at least 1 week following the administration of the oxygenated ionic aqueous solution. In some embodiments, the treatment for impaired respiratory function (such as a ventilator) is deferred at least 1 week, such as at least 2 weeks, 3 weeks, 4 weeks, 5 weeks, 6 weeks, 7 weeks, 8 weeks, 9 weeks, 10 weeks, 11 weeks, or 12 weeks, including ranges between any two of the listed values, such as 1-12 weeks, such as 1-2 weeks, 1-3 weeks, 1-4 weeks, 1-5 weeks, 1-6 weeks, 1-7 weeks, 1-8 weeks, 1-9 weeks, 1-10 weeks, 1-11 weeks, 1-12 weeks, 2-3 weeks, 2-4 weeks, 2-5 weeks, 2-6 weeks, 2-7 weeks, 2-8 weeks, 2-9 weeks, 2-10 weeks, 2-11 weeks, 2-12 weeks, 3-4 weeks, 3-5 weeks, 3-6 weeks, 3-7 weeks, 3-8 weeks, 3-9 weeks, 3-10 weeks, 341 weeks, 3-12 weeks, 4-5 weeks, 4-6 weeks, 4-7 weeks, 4-8 weeks, 4-9 weeks, 4-10 weeks, 4-11 weeks, 4-12 weeks, 5-6 weeks, 5-7 weeks, 5-8 weeks, 5-9 weeks, 5-10 weeks, 5-11 weeks, 5-12 weeks, 6-7 weeks, 6-8 weeks, 6-9 weeks, 6-10 weeks, 6-11 weeks, or 6-12 weeks following the administration of the oxygenated ionic aqueous solution.

In the method of some embodiments, the period of weeks over which the oxygenated ionic aqueous solution is administered comprises at least 4 weeks, for example as at least about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 weeks, including ranges between any two of the listed values. In some embodiments, the period of weeks is at least about 20 weeks. In some embodiments, the period of weeks is at least about 23 weeks. In some embodiments, the period of weeks is about 4-20 weeks, 4-30 weeks, 4-40 weeks, 4-50 weeks, 4-60 weeks, 6-20 weeks, 6-30 weeks, 6-40 weeks, 6-50 weeks, 6-60 weeks, 8-20 weeks, 8-30 weeks, 8-40 weeks, 8-50 weeks, 8-60 weeks, 10-20 weeks, 10-30 weeks, 10-40 weeks, 10-50 weeks, 10-60 weeks, 20-30 weeks, 20-40 weeks, 20-50 weeks, 20-60 weeks, 30-40 weeks, 30-50 weeks, 30-60 weeks, 40-50 weeks, 40-60 weeks, or 50-60 weeks.

In some embodiments, the method further comprises comprising detecting an inhibition or reduction of decline in vital capacity in the patient following administration of the oxygenated ionic aqueous solution. The inhibition or reduction of decline in vital capacity can be detected at least 1, 2, 3, 4, 5, 6, 7, 8, 9, 10, 11, 12, 13,14,15,16, 17, and 19, or 20 weeks after the oxygenated ionic aqueous solution is administered.

In the method of some embodiments, the patient is selected as undergoing the decline in respiratory function. In the method of some embodiments, the patient is selected as being at risk of the decline in respiratory function. In the method of some embodiments, the patient is an ALS patient.

In the method of some embodiments, the decline in respiratory function comprises, consists essentially of, or consists of a decline in vital capacity, for example SVC and/or FVC.

In the method of some embodiments, ameliorating, inhibiting, reducing, treating, slowing the progression of, or preventing the decline of respiratory function (or the rate of decline of respiratory function) comprises reducing a rate at which vital capacity declines, halting a decline in vital capacity, or increasing vital capacity. In the method of some embodiments, ameliorating, inhibiting, reducing, treating, slowing the progression of, or preventing the decline of respiratory function comprises reducing a rate at which vital capacity declines or halting a decline in vital capacity. In the method of some embodiments, the vital capacity comprises SVC and/or FVC.

### Methods of Ameliorating, Inhibiting, Reducing the Symptoms of Treating, Slowing the Progression of, or Preventing a Neurological Disease of a Patient, the Neurological Disease Comprising a Decline in Respiratory Function in a Patient

It has been shown herein that oxygenated ionic aqueous solution in accordance with some embodiments can inhibit the rate of decline of slow vital capacity (SVC), so that the decline in SVC is slower, or there is no decline. See Example 2. Moreover, decline in respiratory function is associated with neurological diseases as described herein, for example, DMD, SMA (type I, II, or III), HD, AD, and PD. Accordingly, it is contemplated that oxygenated ionic aqueous solutions in accordance with some embodiments herein can be used to inhibit, reduce, treat, a slowing in the progression of, or prevent a decline in respiratory function in a patient in need of the same. In some embodiments, a method of ameliorating, inhibiting, reducing the symptoms of, treating, slowing the progression of, or preventing a neurological disease of a patient or a symptom thereof is described. The neurological disease can comprise a decline in respiratory function. The method can comprise administering an amount of oxygenated ionic aqueous solution to the patient effective to ameliorate, inhibit, reduce, treat, slow the progression of, or prevent the decline in respiratory function in the patient. Thus, the method can comprise ameliorating, inhibiting, reducing, treating, slowing the progression of, or preventing the neurological disease or a symptom thereof. In some embodiments, the oxygenated ionic aqueous solution is a pharmaceutical saline solution comprising stabilized oxygen-containing nanostructures, a majority of the nanostructures having a diameter of less than 100 nanometers, in which the pharmaceutical saline solution comprised at least 20 ppm oxygen at the time it was manufactured.

It will be appreciated that wherever a method of ameliorating, inhibiting, reducing the symptoms of, treating, slowing the progression of, or preventing a neurological disease in a patient comprising administering an oxygenated ionic aqueous solution is mentioned, an oxygenated ionic aqueous solution (or pharmaceutical composition comprising the oxygenated ionic aqueous solution) for use in ameliorating, inhibiting, reducing the symptoms of, treating, slowing the progression of, or preventing a neurological disease is expressly contemplated as well.

In some embodiments, the oxygenated ionic aqueous solution is administered repeatedly over a period of weeks. The administering can comprise intravenous administration, inhalation, or intravenous administration and inhalation. In the method of some embodiments, the period of weeks over which the oxygenated ionic aqueous solution is administered comprises at least 4 weeks, for example as at least about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 weeks, including ranges between any two of the listed values. In some embodiments, the period of weeks is at least about 20 weeks. In some embodiments, the period of weeks is at least about 23 weeks. In some embodiments, the period of weeks is about 4-20 weeks, 4-30 weeks, 4-40 weeks, 4-50 weeks, 4-60 weeks, 6-20 weeks, 6-30 weeks, 6-40 weeks, 6-50 weeks, 6-60 weeks, 8-20 weeks, 8-30 weeks, 8-40 weeks, 8-50 weeks, 8-60 weeks, 10-20 weeks, 10-30 weeks, 10-40 weeks, 10-50 weeks, 10-60 weeks, 20-30 weeks, 20-40 weeks, 20-50 weeks, 20-60 weeks, 30-40 weeks, 30-50 weeks, 30-60 weeks, 40-50 weeks, 40-60 weeks, or 50-60 weeks.

In some embodiments, the method comprises selecting the patient as undergoing a decline in respiratory function, or at risk of undergoing a decline in respiratory function. In some embodiments, the method comprises selecting the patient as having, or being at risk of having neurological disease comprising the decline in respiratory function.

As administration of the oxygenated ionic aqueous solution as described herein can ameliorate, inhibit, reduce, treat, slow the progression of, or prevent a decline in respiratory function (or the rate of decline of respiratory function), it is contemplated that following the administration of the oxygenated ionic aqueous solution, a treatment for impaired respiratory function (for example ventilator) can be deferred or avoided. In some embodiments, the method comprises deferring use of a ventilator for the patient for at least 1 week following the administration of the oxygenated ionic aqueous solution. In some embodiments, the treatment for impaired respiratory function (such as a ventilator) is deferred at least 1 week, such as at least 2 weeks, 3 weeks, 4 weeks, 5 weeks, 6 weeks, 7 weeks, 8 weeks, 9 weeks, 10 weeks, 11 weeks, or 12 weeks, including ranges between any two of the listed values, such as 1-12 weeks, such as 1-2 weeks, 1-3 weeks, 1-4 weeks, 1-5 weeks, 1-6 weeks, 1-7 weeks, 1-8 weeks, 1-9 weeks, 1-10 weeks, 1-1.1 weeks, 1-12 weeks, 2-3 weeks, 2-4 weeks, 2-5 weeks, 2-6 weeks, 2-7 weeks, 2-8 weeks, 2-9 weeks, 2-10 weeks, 2-11 weeks, 2-12 weeks, 3-4 weeks, 3-5 weeks, 3-6 weeks, 3-7 weeks, 3-8 weeks, 3-9 weeks, 3-10 weeks, 3-11 weeks, 3-12 weeks, 4-5 weeks, 4-6 weeks, 4-7 weeks, 4-8 weeks, 4-9 weeks, 4-10 weeks, 4-11 weeks, 4-12 weeks, 5-6 weeks, 5-7 weeks, 5-8 weeks, 5-9 weeks, 5-10 weeks, 5-11 weeks, 5-12 weeks, 6-7 weeks, 6-8 weeks, 6-9 weeks, 6-10 weeks, 6-11 weeks, or 6-12 weeks following the administration of the oxygenated ionic aqueous solution.

In the method of some embodiments, the method further comprises comprising detecting an inhibition or reduction of decline in vital capacity in the patient following administration of the oxygenated ionic aqueous solution. The inhibition or reduction of decline in vital capacity can be detected at least 1, 2, 3, 4, 5, 6, 7, 8, 9, 10, 11, 12, 13,14,15,16, 17, and 19, or 20 weeks after the oxygenated ionic aqueous solution is administered.

In the method of some embodiments, the neurological disease comprises at least one of DMD, SMA (type I, II, or III), HD, AD, and PD. In some embodiments, the neurological disease comprises at least one of DMD, SMA (type I, II, or III), or HD. In some embodiments, the neurological disease comprises at least one of DMD, or SMA (type I, II, or III). In some embodiments, the patient does not have ALS.

In the method of some embodiments, ameliorating, inhibiting, reducing, treating, slowing the progression of, or preventing the decline of respiratory function (or the rate of decline of respiratory function) comprises reducing a rate at which vital capacity declines or halting a decline in vital capacity (for example SVC and/or FVC). In the method of some embodiments, the decline in respiratory function comprises, consists essentially of, or consists of a decline in vital capacity. In the method of some embodiments, ameliorating, inhibiting, reducing, treating, slowing the progression of, or preventing the decline of respiratory function (or the rate of decline of respiratory function) comprises reducing a rate at which vital capacity declines, halting a decline in vital capacity, or increasing vital capacity. In the method of some embodiments, ameliorating, inhibiting, reducing, treating, slowing the progression of, or preventing the decline of respiratory function (or the rate of decline of respiratory function) comprises reducing a rate at which vital capacity declines or halting a decline in vital capacity. In the method of some embodiments, the vital capacity comprises SVC and/or FVC.

### Additional Options for Methods

The following options are contemplated in accordance with any of the methods described herein (or their corresponding uses or medicaments), for example, methods of treating, preventing, ameliorating, or reducing the symptoms of ALS as described herein, methods of deferring an ALS intervention in an ALS patient as described herein, and/or methods of ameliorating, inhibiting, reducing, treating, slowing the progression of, or preventing a decline in respiratory function (or the rate of decline of respiratory function) in a patient as described herein.

In some embodiments, for any of the methods described herein (or their corresponding uses or medicaments), the period of weeks over which the oxygenated ionic aqueous solution is administered comprises at least 4 weeks. For example, in methods (or corresponding uses or medicaments) of some embodiments, the oxygenated ionic aqueous solution is administered for a period of weeks that is least about 4 weeks, for example as at least about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 weeks, including ranges between any two of the listed values. In some embodiments, the period of weeks is at least about 20 weeks. In some embodiments, the period of weeks is at least about 23 weeks. In some embodiments, the period of weeks is about 4-20 weeks, 4-30 weeks, 4-40 weeks, 4-50 weeks, 4-60 weeks, 6-20 weeks, 6-30 weeks, 6-40 weeks, 6-50 weeks, 6-60 weeks, 8-20 weeks, 8-30 weeks, 8-40 weeks, 8-50 weeks, 8-60 weeks, 10-20 weeks, 10-30 weeks, 10-40 weeks, 10-50 weeks, 10-60 weeks, 20-30 weeks, 20-40 weeks, 20-50 weeks, 20-60 weeks, 30-40 weeks, 30-50 weeks, 30-60 weeks, 40-50 weeks, 40-60 weeks, or 50-60 weeks. In some embodiments, the period of weeks over which the oxygenated ionic aqueous solution is administered is about 20-50 weeks.

In the methods (or corresponding uses or medicaments) of some embodiments, the oxygenated ionic aqueous solution is administered intravenously at least weekly over the period of weeks, for example weekly for at least 20 weeks. In the methods (or corresponding uses or medicaments) of some embodiments, the oxygenated ionic aqueous solution is administered intravenously only once per week. In the methods (or corresponding uses or medicaments) of some embodiments, the oxygenated ionic aqueous solution is administered intravenously more than once per week. In some embodiments, the oxygenated ionic aqueous solution is administered via inhalation (e.g. nebulization) multiple times a week over the period of weeks, for example for about 20-50 weeks. In methods (or corresponding medicaments or uses) of some embodiments, the oxygenated ionic aqueous solution is administered intravenously at least weekly and via inhalation (e.g., nebulization) multiple times per week (e.g., at least 6 times per week) over the period of 20-50 weeks. By way of example, treating neurological disease (e.g., ALS) patients for at least 23 weeks using oxygenated ionic aqueous solution in accordance with some embodiments herein has been shown to inhibit the rate of vital capacity decline (See Example 2).

In some embodiments, for any of the methods as described herein (or corresponding uses or medicaments), the oxygenated ionic aqueous solution is administered intravenously. In the methods (or corresponding uses or medicaments) of some embodiments, the oxygenated ionic aqueous solution is administered intravenously, in an amount of at least about 100 ml, such as at least 100 ml, 150 ml, 200 ml 250 ml, 300 ml, 350 ml, 400 ml, 450 ml, 500 ml, 600 m, 700 ml, 800 ml, 900 ml, or 1000 ml per intravenous administration, including ranges between any two of the listed values. For example, in methods (or corresponding uses or medicaments) of some embodiments, about 100 ml-2000 ml or a sub-range thereof of the oxygenated ionic aqueous solution is administered intravenously per intravenous administration, for example about 100 ml-1500 ml, 100 ml-1000 ml, 100 ml-900 ml, 100 ml-800 ml, 100 ml-700 ml, 100 ml-500 ml, 100 ml-400 ml, 100 ml-300 ml, 100 ml-200 ml, 200 ml-900 ml, 200 ml-800 ml, 200 ml-700 ml, 200 ml-500 ml, 200 ml-400 ml, 200 ml-300 ml, 300 ml-900 ml, 300 ml-800 ml, 300 ml-700 ml, 300 ml-500 ml, or 300 ml-400 ml per intravenous administration. In methods (or corresponding medicaments or uses) of some embodiments, the oxygenated ionic aqueous solution is administered intravenously in an amount of at least 350 ml per intravenous administration. In methods (or corresponding medicaments or uses) of some embodiments, the oxygenated ionic aqueous solution is administered intravenously in an amount of at least 375 ml per intravenous administration. In methods (or corresponding medicaments or uses) of some embodiments, the oxygenated ionic aqueous solution is administered intravenously in an amount of about 350-400 ml per intravenous administration. In methods of inhibiting, treating, preventing, ameliorating, or reducing the symptom of a neurological disease (e.g., ALS), or of deferring a neurological disease (e.g. ALS) intervention in an neurological disease (e.g., ALS) patient of some embodiments (or corresponding medicaments or uses), the oxygenated ionic aqueous solution is administered intravenously in an amount of about 100 ml-1000 ml per intravenous administration. In methods of some embodiments (or corresponding medicaments or uses), the oxygenated ionic aqueous solution is administered intravenously in an amount of about 200 ml-600 ml per intravenous administration. In methods of some embodiments (or corresponding medicaments or uses), the oxygenated ionic aqueous solution is administered intravenously in an amount of about 300 ml-500 ml per intravenous administration. In some embodiments, for any of the methods as described herein (or corresponding uses or medicaments), in addition to being administered intravenously as described herein, the oxygenated ionic aqueous solution is administered via inhalation as described herein.

In some embodiments, for any of the methods as described herein (or corresponding uses or medicaments), the oxygenated ionic aqueous solution is administered via inhalation. For example, the inhalation can comprise nebulization. For example, the inhalation can comprise a spray. In the methods of some embodiments (or corresponding uses or medicaments as described herein), the oxygenated ionic aqueous solution is administered by inhalation at least 2 times per week over the period of weeks, for example at least 2, 3, 4, 5, 6, 7, 8, 9, 10, 11, 12, 13, or 14 times per week by inhalation, including ranges between any two of the listed values, for example, 2-14 times per week, 2-12 times per week, 2-10 times per week, 2-8 times per week, 2-7 times per week, 3-14 times per week, 3-10 times per week, 3-8 times per week, 3-7 times per week, 4-14 times per week, 4-10 times per week, 4-8 times per week, 4-7 times per week, 5-14 times per week, 5-10 times per week, 5-8 times per week, 5-7 times per week, 6-15 times per week, 6-10 times per week, 6-8 times per week, or 6-7 times per week over the period of weeks. In methods (or corresponding uses or medicaments) of some embodiments, the oxygenated ionic aqueous solution is administered by inhalation at least 6 times per week over the period of weeks. In methods (or corresponding uses or medicaments) of some embodiments, the oxygenated ionic aqueous solution is administered by inhalation 2 to 12 times per week over the period of weeks. In methods (or corresponding uses or medicaments) of some embodiments, the oxygenated ionic aqueous solution is administered by inhalation 4 to 8 times per week over the period of weeks. In some embodiments, for any of the methods as described herein (or corresponding uses or medicaments), in addition to being administered by inhalation as described herein, the oxygenated ionic aqueous solution is administered intravenously as described herein.

The amount of oxygenated ionic aqueous solution that is administered by inhalation (e.g., nebulization or spray) can comprise at least about 2 ml in methods of some embodiments (or their corresponding uses or medicaments). In methods of some embodiments (or their corresponding uses or medicaments), at least 2 ml, 3 ml, 4 ml, 5 ml, 6 ml, 7 ml, 8 ml, 9 ml, 10 ml, 11 ml, 12, ml, 13 ml, 14 ml, 15 ml, 16 ml, 17 ml, 18 ml, 19 ml, or 20 ml of the oxygenated ionic aqueous solution is administered by inhalation, including ranges between any two of the listed values, for example, 1 ml-8 ml, 1 ml-10 ml, 1 ml-20 ml, 2 ml 8 ml, 2 ml-10 ml, 2 ml-20 ml, 3 ml-8 ml, 3 ml-10 ml, 3 ml-20 ml, 4 ml-8 ml, 4 ml-10 ml, or 4 ml-20 ml. In methods of some embodiments (or their corresponding uses or medicaments), the oxygenated ionic aqueous solution is administered by inhalation (e.g., nebulization or spray) in an amount of at least 2 ml. In methods of some embodiments (or their corresponding uses or medicaments), the oxygenated ionic aqueous solution is administered by inhalation (e.g., nebulization or spray) in an amount of at least 4 ml. In methods of some embodiments (or their corresponding uses or medicaments), the oxygenated ionic aqueous solution is administered by inhalation (e.g., nebulization or spray) in an amount of about 1 ml to 10 ml. In methods of some embodiments (or their corresponding uses or medicaments), the oxygenated ionic aqueous solution is administered by inhalation (e.g., nebulization or spray) in an amount of about 2 ml to 8 ml.

In some embodiments, for any of the methods (or corresponding uses or medicaments as described herein), the oxygenated ionic aqueous solution is a pharmaceutical saline solution comprising at least 20 ppm oxygen at the time it was manufactured (such as at least 30 ppm, 40 ppm, 50 ppm, 60 ppm, 70 ppm, or 80 ppm), and is administered intravenously weekly in an amount of at least 200 ml (such as at least 200 ml, 250 ml, 300 ml or 350 ml), and is administered by inhalation comprising nebulization at least 6 times per week in an amount of at least 2 ml (such as at least 1 ml, 2 ml, 3 ml, or 4 ml), and the period of weeks is at least 20 weeks.

In some embodiments, for any of the methods (or corresponding uses or medicaments as described herein), the oxygenated ionic aqueous solution is a pharmaceutical saline solution comprising at least 20 ppm oxygen at the time it was manufactured (such as at least 30 ppm, 40 ppm, 50 ppm, 60 ppm, 70 ppm, or 80 ppm), and is administered intravenously weekly in an amount of at least about 375 ml, and is administered by inhalation comprising nebulization at least 6 times per week in an amount of at least about 4 ml, and the period of weeks is at least 23 weeks. Optionally, the pharmaceutical saline solution can comprise other active or inactive ingredients.

It has been shown herein that treatment with oxygenated ionic aqueous solution in accordance with some embodiments herein can enhance myelination by oligodendrocytes (See Example 3), support or enhancing nerve myelination (See Example 4), decrease motor neuron loss in the spinal cord (See Example 5), preserve neuromuscular junctions (See Example 6), increase protective function of microglia (such as phagocytosis) (See Example 7), activate protective microglia in the central nervous system (e.g., brain and/or spinal cord) (See Example 8), and protect cells harboring a mitochondrial deficit associated with ALS patients (See Example 9). In some embodiments, for any of the methods (or corresponding uses or medicaments) as described herein, inhibiting, treating, preventing, ameliorating, or reducing the symptoms of ALS comprises one or more of: enhancing myelination by oligodendrocytes, enhancing myelination by Schwann cells, supporting or enhancing nerve myelination, decreasing motor neuron loss in the spinal cord, preserving neuromuscular junctions, increasing protective function of microglia (such as phagocytosis), and/or activating protective microglia in the central nervous system (e.g., brain and/or spinal cord). In some embodiments, for any of the methods (or corresponding uses or medicaments) as described herein, the method further comprises detecting prevention, amelioration, and/or reduction of the symptoms of a neurological disease (e.g., ALS) of the subject after treatment with the oxygenated ionic aqueous solution for the period of weeks.

In some embodiments, for any of the methods (or corresponding uses or medicaments) as described herein, inhibiting, treating, preventing, ameliorating, or reducing the symptoms of a neurological disease (e.g., ALS) comprises one or more of reduction of peripheral inflammation (effector T cells down and/or regulatory T cells up), reduction of CNS inflammation (decrease in inflammatory glia and/or reduction of NFkB activity), increase in non-inflammatory microglia, increase in survival, maturation and function of oligodendrocytes, augmentation of neuronal branching, plasticity and neurotransmission, enhancement of neuronal survival (anti-apoptotic), and/or stimulation of mitochondrial biogenesis and function. In some embodiments, any of the methods (or corresponding uses or medicaments) as described herein, further comprises detecting at least one of: reduction of peripheral inflammation (effector T cells down and/or regulatory T cells up), reduction of CNS inflammation (decrease in inflammatory glia and/or reduction of NFkB activity), increase in non-inflammatory microglia, increase in survival, maturation and function of oligodendrocytes, augmentation of neuronal branching, plasticity and neurotransmission, enhancement of neuronal survival (anti-apoptotic), and/or stimulation of mitochondrial biogenesis and function. In some embodiments, a patient is selected as having impaired mitochondrial biogenesis and/or function.

### EXAMPLE 1

### Stability of Oxygenated Ionic Aqueous Solutions

Oxygenated ionic aqueous solution was generated using a Mixing Device as described in U.S. Pat. No. 9,745,567. The solution was used to manufacture drug product, a solution that contains charge-stabilized oxygen gas-containing nanostructures having an average diameter of less than 100 nanometers.

For this example lot of drug product, bulk drug substance was manufactured in bulk, then filled into drug product containers, and drug product was prepared. At the time that the drug product of this Example was prepared, (e.g., filled in syringes, IV bags, or glass vials) its dissolved oxygen content was spec'd at greater than or equal to 50 ppm.

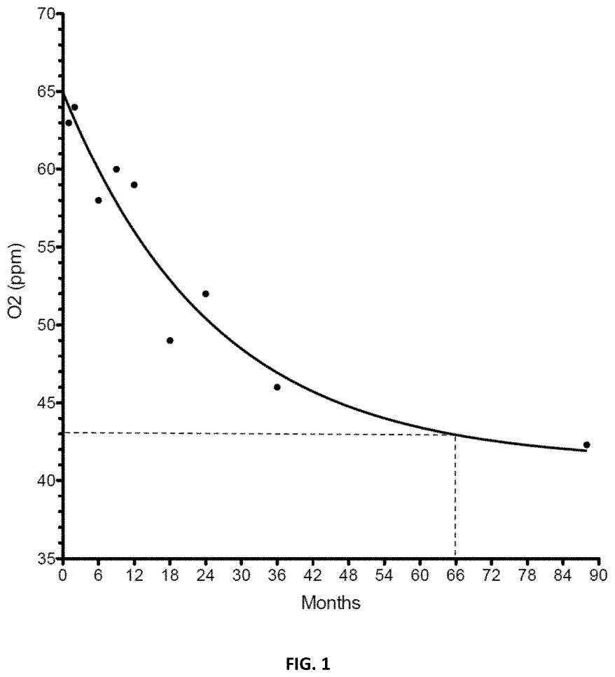

Lot release testing was done for this example lot of drug product. Extended testing of oxygen levels was performed to establish a DO profile over time. FIG. 1 shows a curve obtained for lot stability over time. At 66 months after filling, this lot had a DO content of about 43 ppm.

At 66 months, the oxygenated ionic aqueous saline solution from this lot was collected, and tested for anti-inflammatory activity in a mouse model of inflammation. Female SJL/j mice were immunized with MBP and on day 10 of immunization, spleens and lymph nodes were harvested, followed by treatment of splenocytes and lymph node cells in the presence of 5% and 10% oxygenated ionic aqueous solution drug product from the lot (66 months after filling) or one of its controls or processing variants. After 24 hours, mRNA expression of Foxp3 and IL-10 as well as other related markers of Treg, Th17, Th1 and Th2 were measured as reported earlier (Mondal 2013). Oxygenated ionic aqueous solution drug product from the lot, with a DO level as low as 43 ppm, showed the activities of reversing MBP-induced reduction in IL-10 and the Treg marker FoxP3.

Thus, it has been shown that oxygenated ionic aqueous solution comprising saline and having at least 50 ppm dissolved oxygen at the time of manufacture (in accordance with methods, uses, and medicaments of some embodiments herein) can be successfully produced, and can stably maintain dissolved oxygen levels for at least 66 months. Moreover, after 66 months, the oxygenated ionic aqueous solution retained anti-inflammatory activity in vivo.

### EXAMPLE 2

### Clinical Studies of Oxygenated Ionic Aqueous Solution on ALS

Phase I safety studies of the oxygenated ionic aqueous solution RNS60 (having a dissolved oxygen content of at least 50 ppm at the time of manufacture) were completed for intravenous (IV) and inhalation (nebulization) dosing to healthy human subjects, and there were no treatment-related serious adverse events (SAEs). In the examples herein, “RNS60” refers to an oxygenated ionic aqueous solution comprising standard saline and a dissolved oxygen content of at least 50 ppm at the time of manufacture.

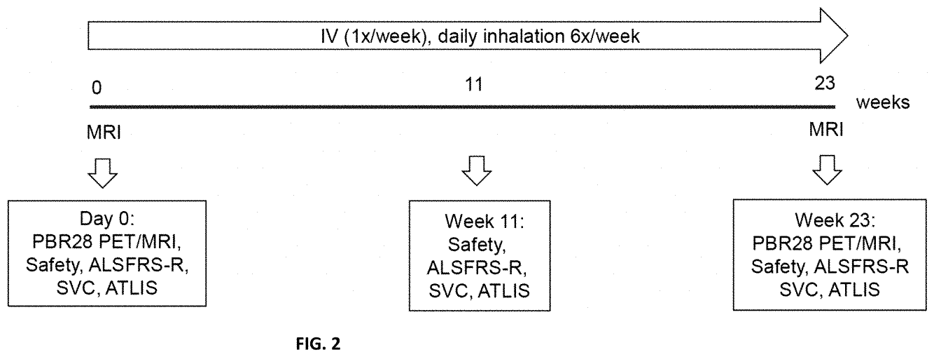

Furthermore, an open-label Phase IIa study was performed in ALS patients, testing the effects of prolonged IV and inhaled administration of the RNS60. The testing was performed on 16 patients. Each patient was treated with 375 ml of RNS60 intravenously once per week, and 4 nil RNS60 by inhalation (nebulization) six times per week. Safety, ALSFRS-R, SVC, and ATLIS (strength) were measured at Day 0. Safety, ALSFRS-R, SVC, and ATLIS were measured at Week 11. Safety, ALSFRS-R, SVC, and ATLIS were measured at Week 23. Additionally, blood biomarkers were measured, and in a 10-patient subgroup, PBR28 PET/MRI was performed as an inflammation biomarker at Day 0 and once more between Week 18 and Week 23. The timeline is summarized schematically in FIG. 2.

In the Phase IIa study, there were no treatment-related SAEs. Furthermore, there were no negative changes in vital signs or in laboratory tests. Furthermore, no participant withdrew due to adverse events (AEs). RNS60 was well-tolerated.

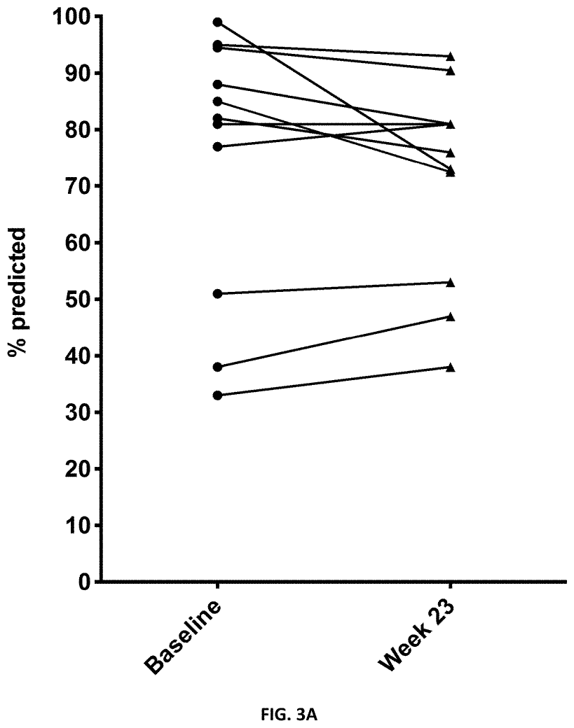

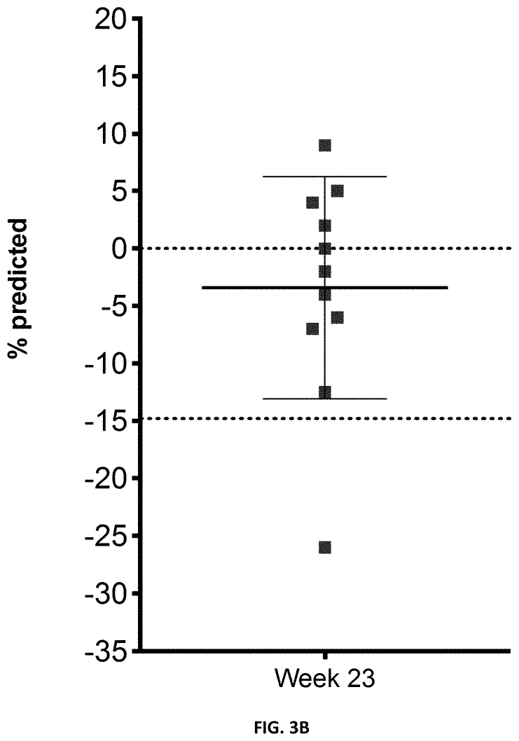

In the RNS60-treated ALS patients, SVC declined at a rate that was about 5-fold less than expected. Vital capacity is an indicator of survival and disease progression in ALS patients (See, e.g, Czaplinski et al., J Neurol Neurosurg Psychiatry 2006). Given reported decline rates for ALS patients, the average SVC drop over 23 weeks was expected to be ˜15 percentage points, whereas the observed average decline was ˜3 percentage points. Overall, there was an ˜80% reduction in the average rate of decline, and remarkably, 5 out of 11 patients showed no decline over 23 weeks. Consistent with a nexus between the reduction in SVC and improved outcomes in ALS patients, a study by Andrews et al., (JAMA Neurol. 2018) predicts that reducing the rate of SVC decline affects ALS disease milestones including onset of respiratory insufficiency, tracheostomy, and death. The SVC level scores (as % of predicted) are shown in FIG. 3A. SVC change from baseline is shown in FIG. 3B. As summarized in Table 1 below, RNS60 dramatically slowed. SVC decline. While the decline of ALSFRS-R was somewhat lower than the average monthly decline of −1.02 points reported from the PROACT database (Atassi et al., 2014), ATLIS scores were not impacted, and the study was not powered to detect any effect on these additional endpoints. The somewhat lower decline in ALSFRS-R scores (a functional rating scale that incorporates assessments of respiratory function, along with assessments of other aspects) in patients treated with RNS60 is consistent with, and supportive of the inhibition in the decline of SVC that is observed for RNS60 treatment.

TABLE 1

| | Change per month | Change after 23 weeks |
|---|---|---|
| | (mean ± | (mean ± |
| Outcome | standard deviation) | standard deviation) |
| Slow Vital Capacity (SVC)* | −0.6 ± 4.41 | −3.3 ± 10.16 |
| ALSFRS-R Total Score | −0.9 ± 0.52 | −4.6 ± 2.83 |
| ATLIS Arm Measures* | −1.7 ± 3.12 | −9.0% ± 7.19 |
| ATLIS Leg Measures* | −1.7 ± 5.61 | −9.1% ± 12.91 |

*Percentage points

Thus, the clinical data show that oxygenated ionic aqueous solutions administered to ALS patients in accordance with some embodiments herein dramatically inhibits the rate of decline in SVC.

The lower SVC decline in patients treated with RNS60 is consistent with the following preclinical findings. This includes prevention of motor neuron loss in ALS mice (See Vallarola et al., J Neuroinflam 2018); preservation of neuromuscular innervation in ALS mice (See Vallarola et al., J Neuroinflam 2018); protection of nerve myelination in ALS mice (See Vallarola et al., J Neuroinflam 2018); and improvement of myelination by oligodendrocytes in vitro (See Rao et al., Scientific Reports 2016 and Jana et al., Neurochem Res 2018).

Other trends in the Phase IIa clinical trial corroborated anti-inflammatory effects of the RNS60 observed in pre-clinical studies. These are summarized in Table 2, below.

TABLE 2

| Clinical | | |
|---|---|---|
| Signal | Preclinical Data | References |
| Greater | Expansion of protective | Vallarola et al., J Neuroinflam |
| PBR28 | microglia in ALS mice | 2018 |
| uptake | Promotion of protective | V. Rao, J. Antel; |
| | microglia in vitro | unpublished data |
| Decreased | Suppression of IL-17 | Mondal et al., PLoS ONE 2012; |
| IL-17 | and Th17 cells in | Mondal et al., |
| | mouse MS model | Neurochem Res 2017 |
| Maintained | Increased Treg | Vallarola et al., J Neuroinflam |
| number of | numbers in ALS & | 2018; |
| Tregs | MS (EAE) mice | Mondal et al., PLoS ONE 2012; |
| | | Mondal et al., Neurochem |
| | | Res 2017 |

Thus, the clinical data show that oxygenated ionic aqueous solutions administered to ALS patients in accordance with some embodiments herein has anti-inflammatory effects, which correlate to anti-inflammatory effects Observed in previous pre-clinical studies.

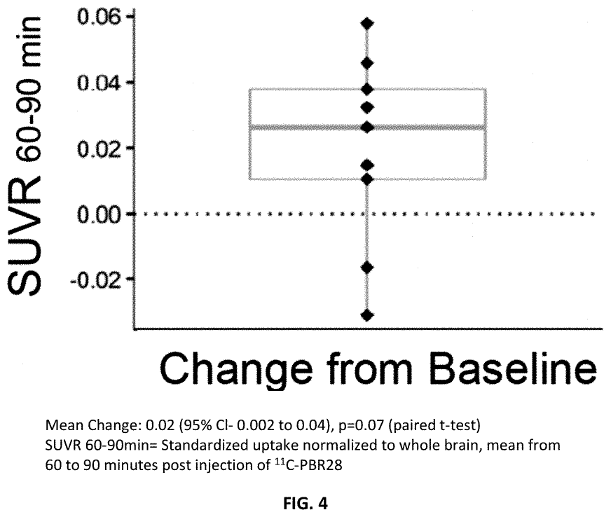

PET imaging was further performed on selected biomarkers. ¹¹C-PBR28 binds to activated glial cells and shows higher signal in the motor cortex of ALS patients. PBR28 does not distinguish between inflammatory and protective glia. In the study, 7 out of 9 patients showed an increase in PBR28 signal from baseline (FIG. 4). The observed increase mirrors findings in ALS mice, where the therapeutic benefits of RNS60 included an increase in protective astroglia and microglia (Vallarola et al., J Neuroinflam 2018).

In the Phase IIa study, levels of blood biomarkers were also measured. The results are summarized in Table 3, below.

TABLE 3

| | | | | FoxP3 | |
|---|---|---|---|---|---|
| | | IL-17 | | (rel. RNA | |
| Visit | N | (pg/mL) | P value | level) | P value |
| T1 (Baseline) | 10 | 86.429 | | 2.509 | |
| T2 (Week 11) | 9 | 71.216 | 0.320 | 2.781 | 0.457 |
| T3 (Week 23) | 8 | 62.996 | 0.293 | 2.493 | 0.964 |

Levels of IL-17, a pro-inflammatory cytokine (typically upregulated in ALS patients; See Fiala et al., J Neuroinflam 2010), showed a downward trend. Levels of FoxP3+Tregs, which are protective in ALS (See Sheean et al., JAMA Neurol 2018), remained stable. Thus, treatment of ALS subjects with oxygenated ionic solution in accordance with some embodiments herein produces a downward trend in inflammation, as measured by the biomarkers IL-17 and FoxP3.

### EXAMPLE 3

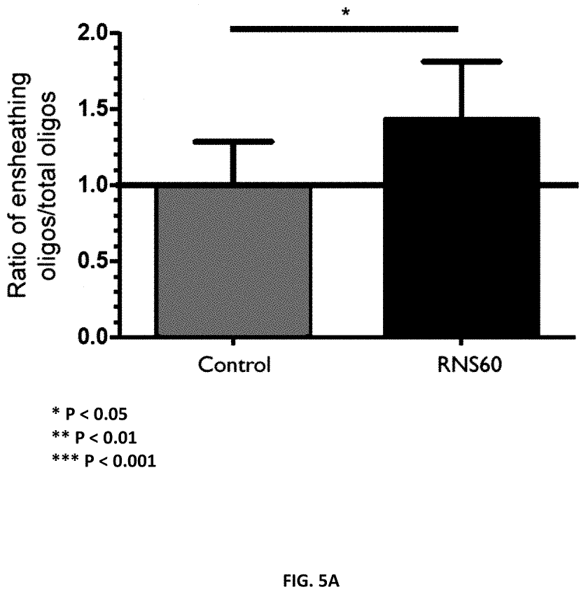

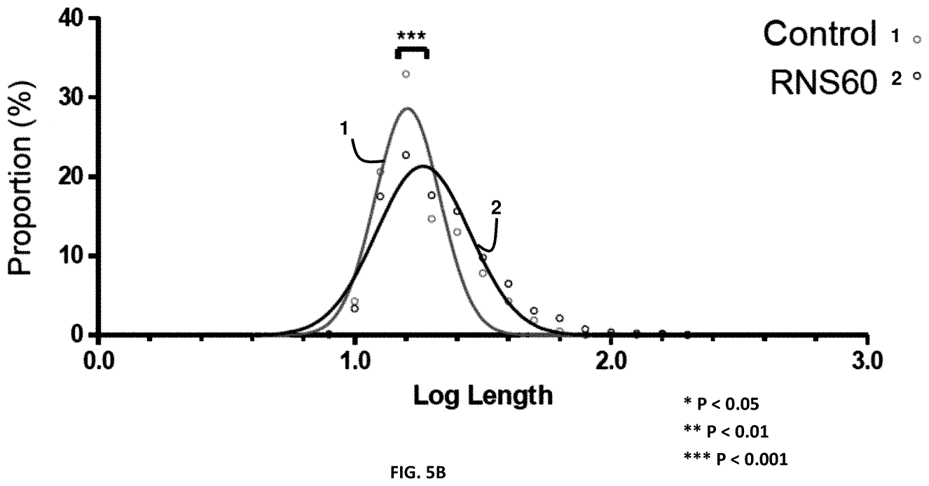

It was observed that RNS60 improves myelination by oligodendrocytes (in vitro). An in vitro analysis was performed using a model of myelination using nanofibers. RNS60 increased the number of ensheathing oligodendrocytes. RNS60 also increased the number of ensheathing processes per oligodendrocyte (See FIG. 5A). Remarkably, RNS60-treated oligodendrocytes 2 had longer ensheathing processes than controls 1 (See FIG. 5B), indicating improved myelination. This is consistent with additional work, which showed that RNS60 directly supports survival and differentiation of oligodendrocytes through bioenergetic support (Rao et al., Scientific Reports 2016).

### EXAMPLES 4-6

Examples 4-6 refer to a study of RNS60 in a mouse model of ALS. Transgenic animals (B6SJL-TgN SOD-1-SOD1^{G93A-1Gur}) were originally obtained from Jackson laboratories (USA) and then maintained on a C57BL6/J (following indicated as C57BL/6-SOD1^{G93A} or referred to as “ALS mice”) at the Mario Negri Institute for Pharmacological Research, Milan, Italy (IRFMN). The animals were housed under SPF (specific pathogen free) standard conditions (22±1° C., 55±10% relative humidity and 12-h light/dark schedule), 3-4 per cage, with free access to food (standard pellet, Altromin, MT, Rieper) and water.

Female C57BL/6-SOD1^{G93A} mice were treated every other day by intraperitoneal (i.p.) injection with 300 μl of RNS60 or Normal Saline (NS) Treatments of SOD1^{G93A} mice (22 mice per group) started from the onset of the disease evaluated when the body weight is at the peak level and the hind limbs show first signs of tremors and reduced abduction but not impairment in motor performance and muscle strength. This occurs at 15 weeks of age in the mouse colony; therefore, all mice start the treatment at 105 days. A set of mice (8 per group) were sacrificed at 20 weeks of age (symptomatic stage) for histopathological, biochemical and molecular analysis. The rest of each group (n=14) were followed until the end stage to assess the effect on survival.

### EXAMPLE 4

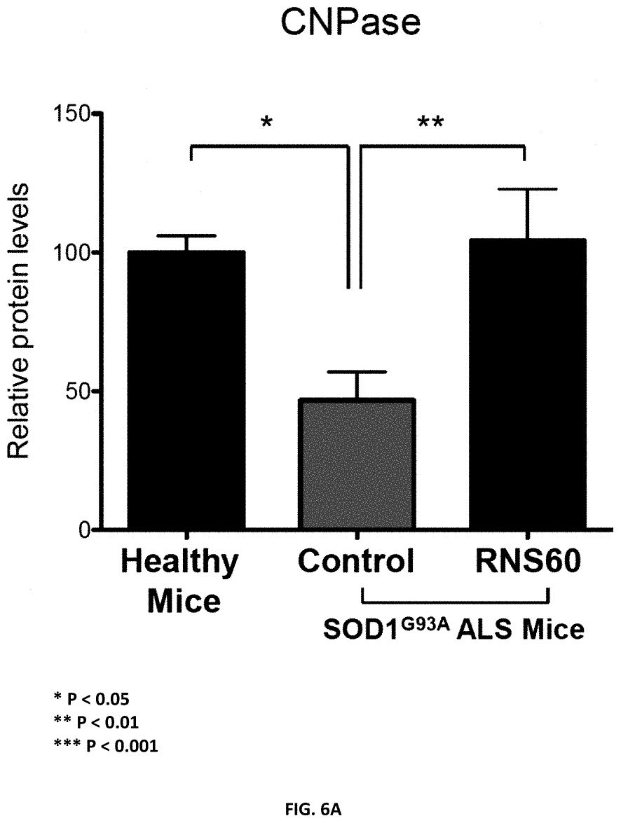

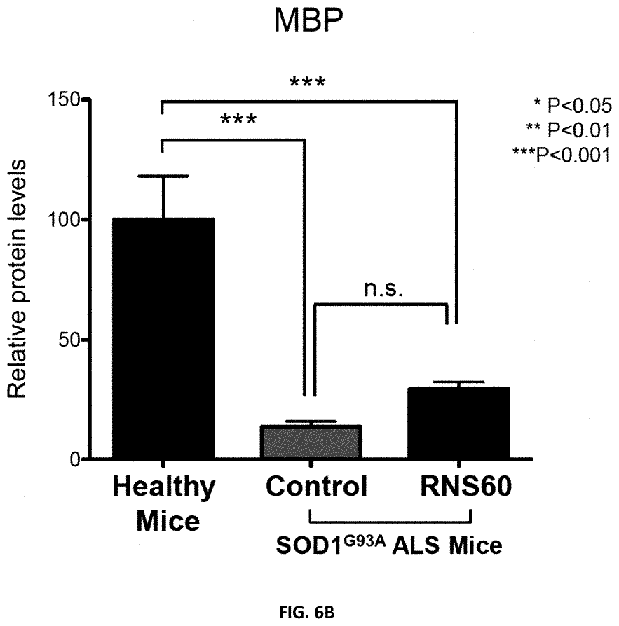

RNS60 was shown to support nerve myelination in ALS mice. CNPase and MBP are components of myelin. As shown in FIG. 6A, treatment of ALS mice with RNS60 significantly increased CNPase protein levels compared to normal saline/placebo treated ALS mouse controls (P<0.01). RNS60 increased levels of MBP relative to controls, but the increase was not statistically significant FIG. 6B.

### EXAMPLE 5

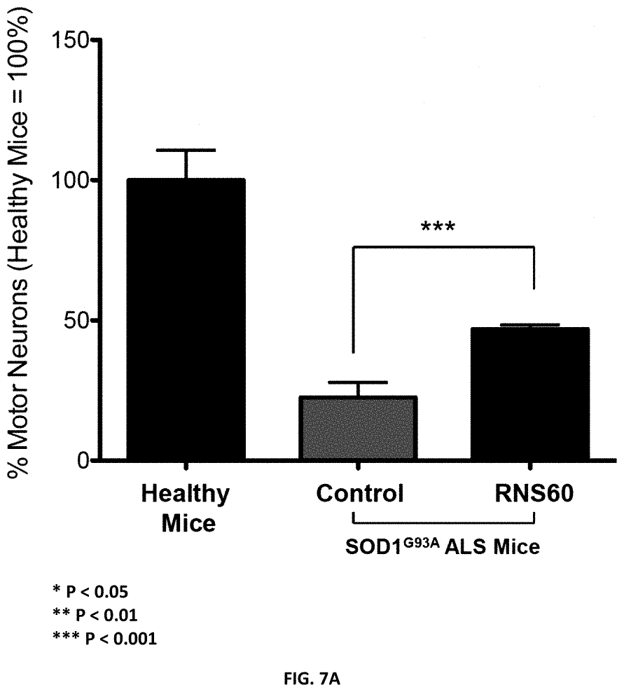

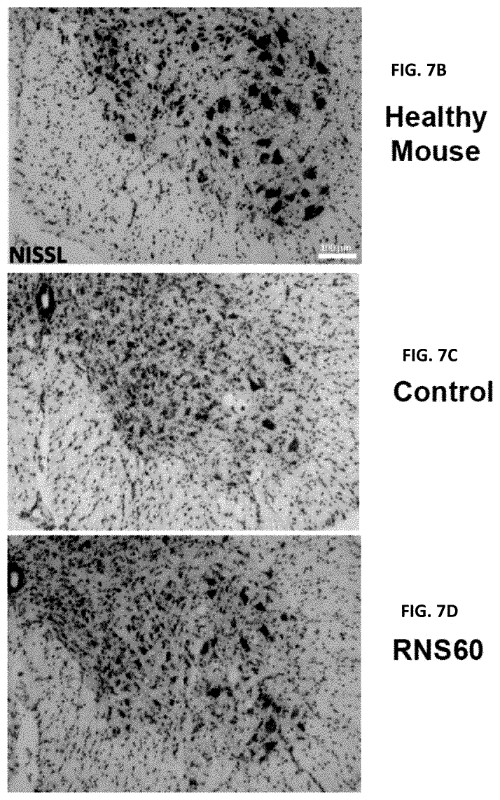

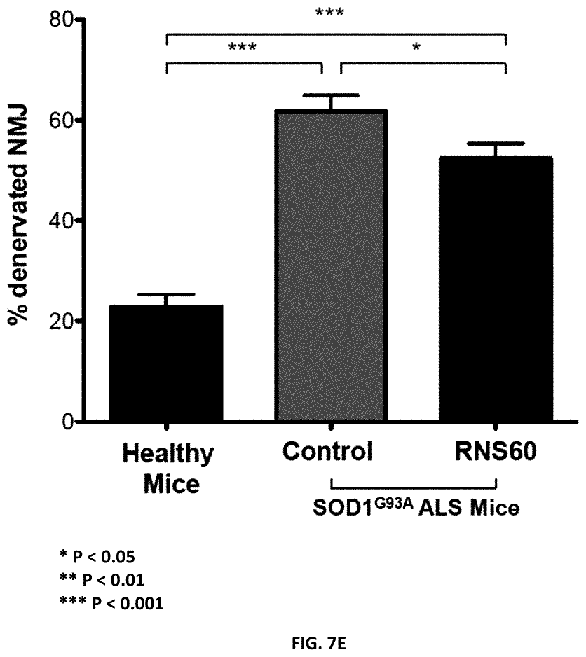

RNS60 was shown to decrease motor neuron loss in the spinal cord of ALS mice. As shown in FIG. 7A, the percentage of motor neurons in ALS mice treated with RNS60 was significantly higher (P<0.001) than in normal saline/placebo treated control ALS mice. FIGS. 7B-7D are a series of microscope images, which show a higher number of motor neurons in RNS60-treated ALS mice (FIG. 7D) than in untreated control ALS mice (FIG. 7C).

### EXAMPLE 6

RNS60 was shown to preserve NMJ in ALS mice. As shown in FIG. 7E, the percentage of denervated NMJ was significantly lower (P<0.05) in RNS60-treated ALS mice than in normal saline/placebo treated control ALS mice.

### EXAMPLES 4-6

### Summary

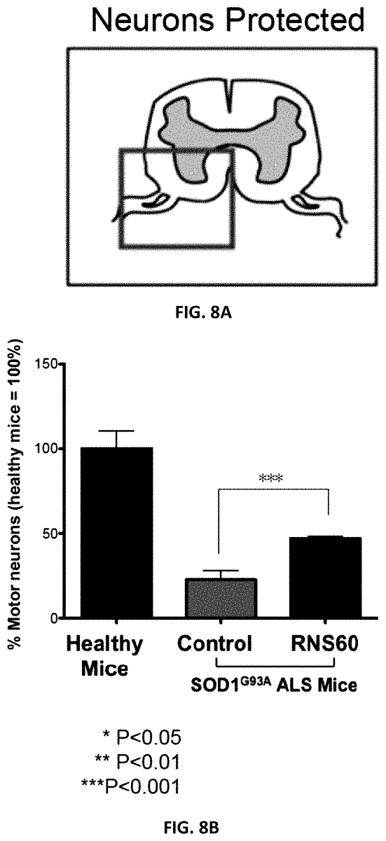

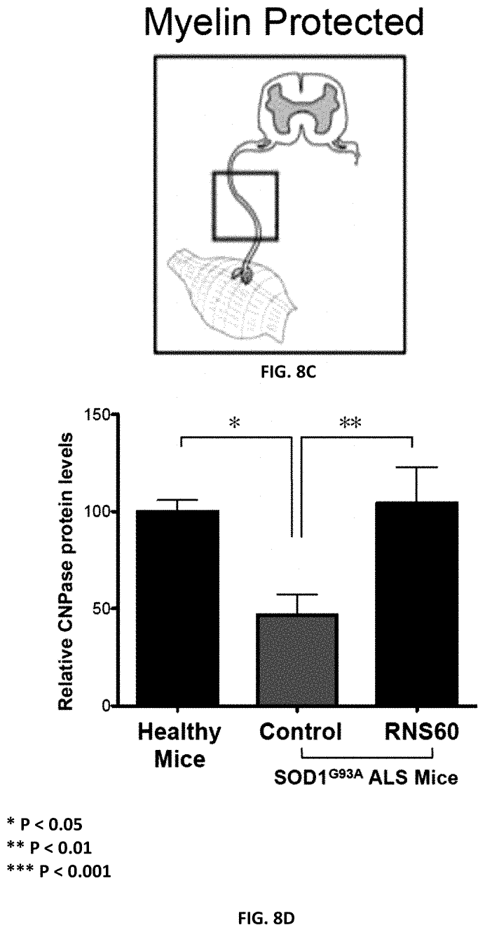

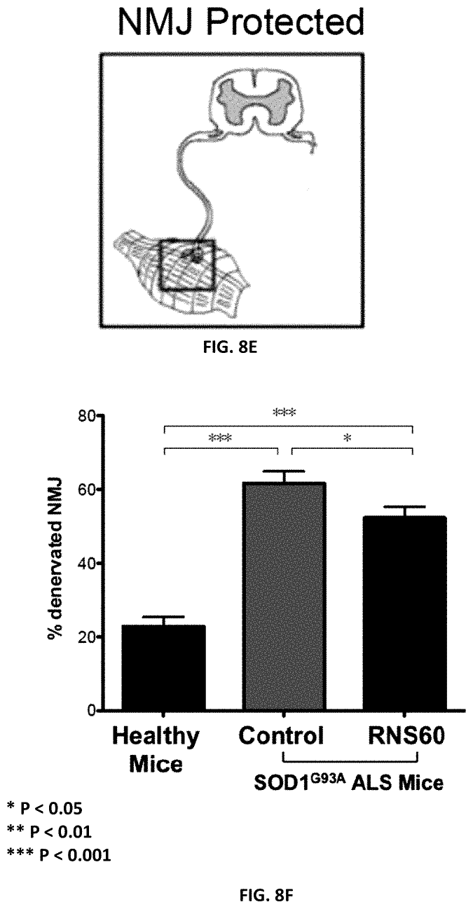

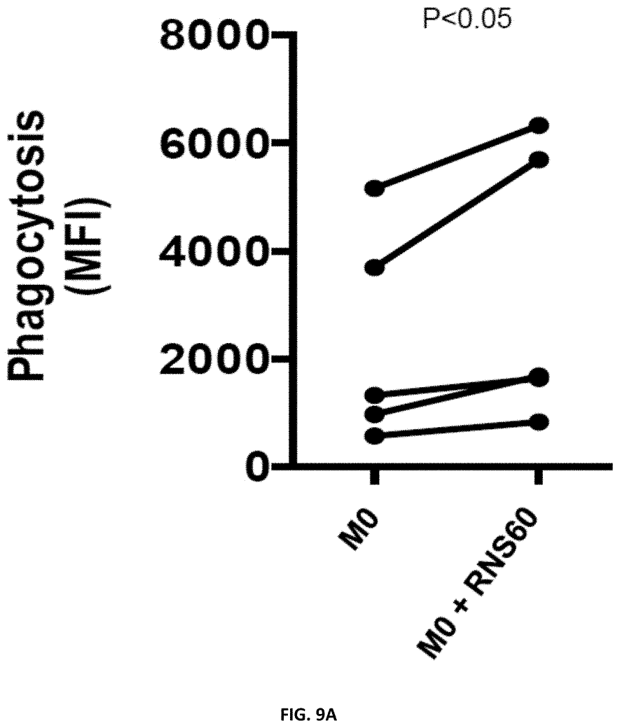

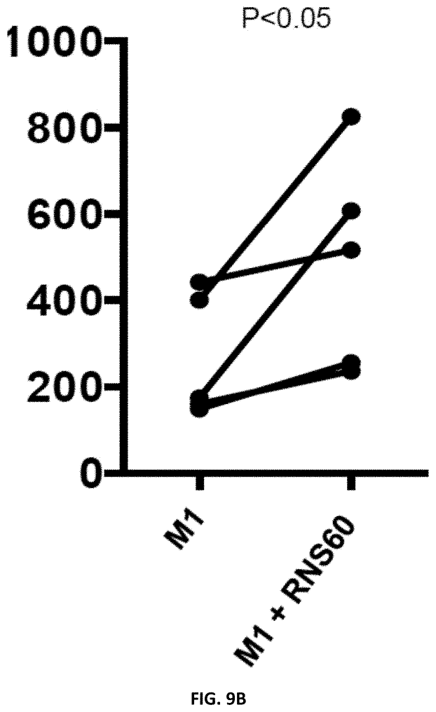

In summary of Examples 4-6, RNS60 was shown to protect multiple components of the neuromuscular system in ALS mice, including protecting motor neurons, protecting myelination, and protecting NMJ (FIGS. 8A-8F). Thus, it is shown that in vivo treatment of an ALS with an oxygenated ionic aqueous solution in accordance with some embodiments herein protects multiple components of the neuromuscular system.

### EXAMPLE 7

It was shown RNS60 increases protective function of microglia (in vitro). In particular, RNS60 increases microglial phagocytosis (removing damaged myelin debris), a significant housekeeping function that plays a noteworthy role in neuronal health. As shown in FIG. 5A, phagocytosis was significantly higher (P<0.05) in undifferentiated microglia (“MO”) treated with RNS60 than in undifferentiated microglia alone. As shown in FIG. 5B, phagocytosis was also significantly higher in inflammatory microglia (“M1”) treated with RNS60 than in inflammatory microglia alone.

Thus, it is shown that in vitro treatment of microglia with an oxygenated ionic aqueous solution in accordance with some embodiments herein increases the protective function of microglia.

### EXAMPLE 8

It was shown that RNS60 activates protective microglia in the spinal cord of ALS mice.

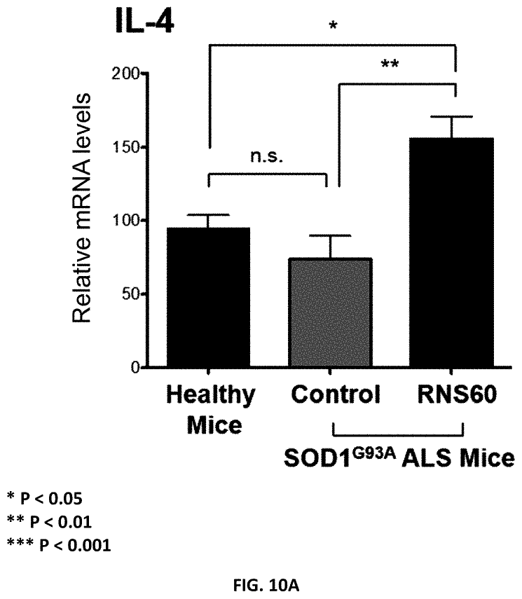

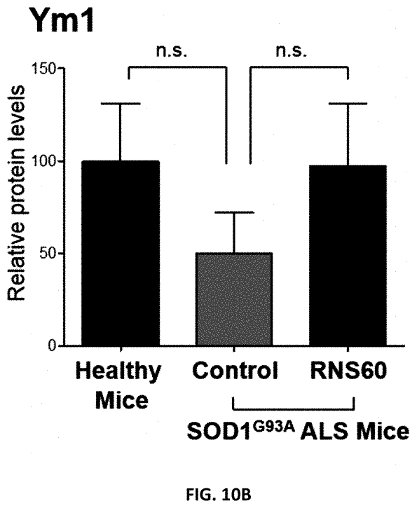

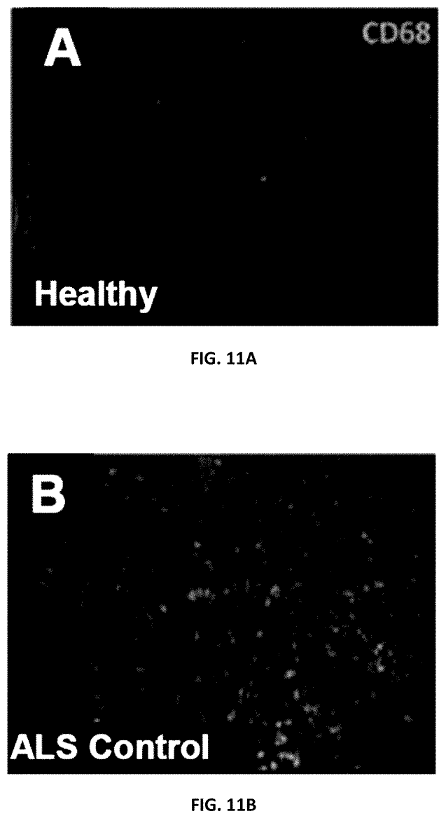

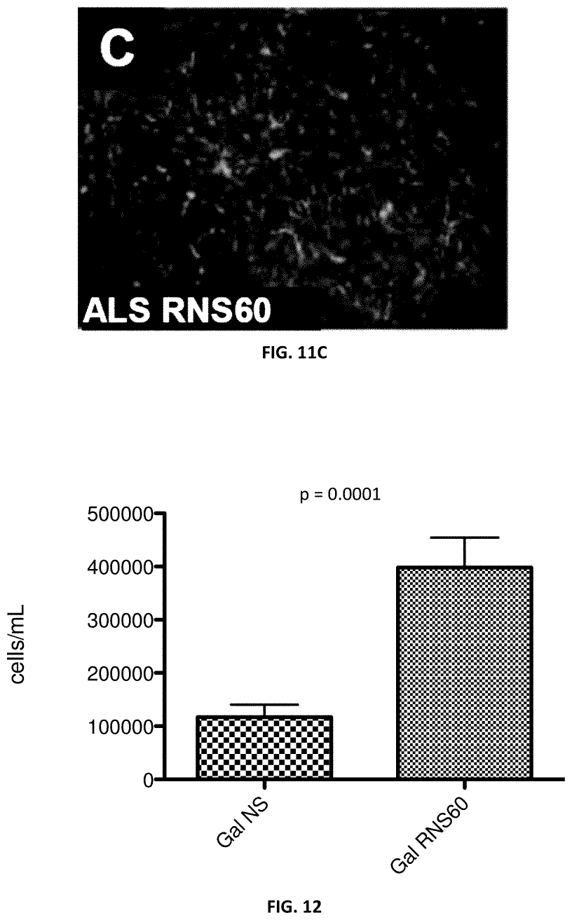

RNS60 treatment increased levels of 1L-4 (a protective cytokine) (FIG. 10A), and prevented a drop in Ym1, a marker of protective (M2) glia (FIG. 10B). Furthermore, in RNS60-treated ALS mice, debris-cleaning microglia were increased (FIG. 11C) compared to untreated control ALS mice (FIG. 11B). Fewer debris-cleaning microglia were observed in healthy mice (FIG. 11A).

Thus, it is shown that in vivo treatment of ALS mice with an oxygenated ionic aqueous solution in accordance with some embodiments herein activates protective microglia in the spinal cord.

### EXAMPLE 9

To directly investigate the effects of the oxygenated ionic aqueous solution RNS60 on mitochondrial function in human cells, cells derived via induced pluripotent stem cell (iPSC) technology from an ALS patient carrying the R15L mutation in the CHCHD10 gene were used. CHCHD10 encodes a protein in the inner mitochondrial membrane that is involved in the regulation of oxidative phosphorylation (OXPHOS (Straub et al. “Loss of CHCHD10-CHCHD2 complexes required for respiration underlies the pathogenicity of a CHCHD10 mutation in ALS.” Hum Mol Genet 2018, 27(1):178-189). Autosomal dominant mutations in CHCHD10 have been identified in sporadic and familial ALS patients as a possible cause for the disease (Cozzolino et al., “Mitochondrial dynamism and the pathogenesis of Amyotrophic Lateral Sclerosis.” Front Cell Neusci 2015, 9, doi: 10.3389/fncel.2015.0003; Imai et al., “Twin CHCH Proteins, CHCHD2, and CHCHD10: Key Molecules of Parkinson's Disease, Amyotrophic Lateral Sclerosis, and Frontotemporal Dementia.” Int J Mol Sci. 2019, 20(4): doi: 10.3390/ijms20040908). Accordingly, the CHCHD10 cell line is a relevant model of impaired mitochondrial function in an ALS context. CHCHD10 R15L mutant cells were cultured in medium containing galactose, which forces them to rely almost entirely on OXPHOS for ATP production, and causes slow growth and increased apoptosis.

In particular, cells were derived from an ALS patient carrying the CHCHD10 R15L mutation using iPSC technology. The cells were plated and cultured in optimal medium for 3 days and then transferred to galactose medium. RNS60 or normal saline (NS, 10% by volume) was added to the medium 24 hours prior to transferring the cells to galactose medium. Cells were cultured in galactose medium for an additional 4 days with daily changes of 10% RNS60 or 10% NS containing medium, and surviving cells were counted. When treated with RNS60, however, a significantly higher number of the cells survived in galactose medium when compared to NS treated cells (FIG. 12), demonstrating a pro-survival effect of RNS60 on cells from an ALS patient with a mitochondrial deficit.

The data in this Example are supportive of, and consistent with, the effectiveness of oxygenated aqueous saline solutions on protecting cells of ALS patients and inhibiting the rate of decline in respiratory function (e.g., vital capacity such as SVC) in an ALS context, as described herein. Thus, these data are supportive of, and consistent with oxygenated aqueous saline solutions ameliorating, inhibiting, reducing, treating, slowing the progression of, or preventing a decline in respiratory function (e.g., vital capacity, such as SVC) in accordance with some embodiments herein.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods, compositions, kits, and uses described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Wherever a method is disclosed herein, for example a method of inhibiting, treating, preventing, ameliorating, or reducing the symptoms of a neurological disease (e.g., ALS), or a method of deferring a neurological disease (e.g., ALS) intervention in a neurological disease (e.g., ALS) patient is disclosed herein, the corresponding use, or composition or medicament for use is also expressly contemplated. For example, for the disclosure of “a method of inhibiting, treating, preventing, ameliorating, or reducing the symptoms of a neurological disease (e.g., ALS) comprising administering an oxygenated ionic aqueous solution,” also contemplated is an oxygenated ionic aqueous solution for use in of inhibiting, treating, preventing, ameliorating, or reducing the symptoms of a neurological disease (e.g., ALS). For example, for the disclosure of a method of deferring a neurological disease intervention in a neurological disease patient comprising administering an oxygenated ionic aqueous solution, also contemplated is an oxygenated ionic aqueous solution for use in treating, preventing, ameliorating, or reducing the symptoms of the neurological disease, the use comprising deferring the neurological disease intervention in the neurological disease.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

1. A method of ameliorating, inhibiting, reducing, treating, slowing the progression of, or preventing a decline in respiratory function or the rate of decline of respiratory function associated with a neurological disease of a patient, the method comprising:
selecting the patient as having the neurological disease and undergoing a decline in respiratory function, having the neurological disease and at risk of a decline in respiratory function, at risk of the neurological disease and undergoing a decline in respiratory function, or at risk of the neurological disease and at risk of undergoing a decline in respiratory function; and
administering an effective amount of oxygenated ionic aqueous solution to the patient, thereby ameliorating, inhibiting, reducing, treating, slowing the progression of, or preventing the decline in respiratory function associated with the neurological disease.

2. The method of claim 1, wherein the oxygenated ionic aqueous solution is a pharmaceutical saline solution comprising stabilized oxygen-containing nanostructures, a majority of the nanostructures having a diameter of less than 100 nanometers, wherein the pharmaceutical saline solution comprised at least 20 ppm oxygen at the time it was manufactured.

3. The method of claim 1, wherein the neurological disease is selected from the group consisting of amyotrophic lateral sclerosis (ALS), Duchenne's muscular dystrophy (DMD), spinal muscular atrophy (SMA) (such as SMA type I, SMA type II, or SMA type III), Huntington's disease, Alzheimer's disease (AD), and Parkinson's disease (PD).

4.-6. (canceled)

7. The method of claim 1, wherein the oxygenated ionic aqueous solution is administered to the subject repeatedly over a period of weeks, said administering comprising intravenous administration, inhalation, or intravenous administration and inhalation.

8. The method of claim 1, further comprising deferring use of a ventilator for the patient for at least 1 week following said administering.

9. The method of claim 8, wherein in the absence of said administering, the ventilator would have been expected at a time, and wherein the ventilator is deferred beyond the expected time.

10. (canceled)

11. The method of claim 1, further comprising detecting an inhibition or reduction or decline in vital capacity in the patient following said administration.

12. (canceled)

13. The method of claim 1, wherein ameliorating, inhibiting, reducing, treating, slowing the progression of, or preventing the decline of respiratory function or the rate of decline of respiratory function comprises reducing a rate at which vital capacity declines, halting a decline in vital capacity, or increasing vital capacity.

14.-19. (canceled)

20. A method of ameliorating, inhibiting, reducing the symptoms of, treating, slowing the progression of, or preventing a neurological disease of a patient or a symptom thereof, the neurological disease comprising a decline in respiratory function, the method comprising:
administering an amount of oxygenated ionic aqueous solution to the patient effective to ameliorate, inhibit, reduce, treat, slow the progression of, or prevent the decline in respiratory function (or the rate of decline of respiratory function) in the patient,
thereby ameliorating, inhibiting, reducing, treating, slowing the progression of, or preventing the neurological disease.

21. The method of claim 20, wherein the oxygenated ionic aqueous solution is a pharmaceutical saline solution comprising stabilized oxygen-containing nanostructures, a majority of the nanostructures having a diameter of less than 100 nanometers, wherein the pharmaceutical saline solution comprised at least 20 ppm oxygen at the time it was manufactured.

22.-24. (canceled)

25. The method of claim 20, further comprising deferring use of a ventilator for the patient for at least 1 week following said administering.

26. The method of claim 25, wherein in the absence of said administering, the ventilator would have been expected at a time, and wherein the ventilator is deferred beyond the expected time.

27. (canceled)

28. The method of claim 20, wherein the neurological disease is selected from the group consisting of amyotrophic lateral sclerosis (ALS), Duchenne's muscular dystrophy (DMD), spinal muscular atrophy (SMA) (such as SMA type I, SMA type II, or SMA type III), Huntington's disease, Alzheimer's disease (AD), and Parkinson's disease (PD).

29.-41. (canceled)

42. A method of deferring an ALS intervention in an ALS patient, the method comprising:
identifying the ALS patient as in need of the ALS intervention at a future time if the patient is untreated with an oxygenated ionic aqueous solution;
administering the oxygenated ionic aqueous solution to the ALS patient repeatedly over a period of weeks, said administering comprising intravenous administration, inhalation, or intravenous administration and inhalation; and
deferring the ALS intervention beyond the future time by at least 1 week.

43. The method of claim 42, further comprising detecting an inhibition of decline in vital capacity in the ALS patient following said administration.

44. The method of claim 42, wherein the ALS intervention comprises a ventilator.

45. The method of claim 42, wherein the ALS intervention comprises a tracheotomy.

46. The method of claim 42, wherein the oxygenated ionic aqueous solution is a pharmaceutical saline solution comprising stabilized oxygen-containing nanostructures, a majority of the nanostructures having a diameter of less than 100 nanometers, wherein the pharmaceutical saline solution comprised at least 20 ppm oxygen at the time it was manufactured.

47. (canceled)

48. (canceled)

49. The method of claim 42, further comprising ameliorating, inhibiting, reducing, treating, slowing the progression of, or preventing a decline in respiratory function (or a rate of decline of respiratory function) in the patient.

50.-68. (canceled)

69. The method of claim 1, wherein the oxygenated ionic aqueous solution is administered intravenously weekly over the period of weeks.

70.-115. (canceled)
